(19) United States
(12) Patent Application Publication
(10) Pub. No.: US 2009/0005711 A1
(43) Pub. Date: Jan. 1, 2009

(54) Systems and methods for opening of the blood-brain barrier of a subject using ultrasound

(76) Inventors: Elisa E. Konofagou, New York, NY (US); James J. Choi, Englewood, NJ (US); Mathieu Pernot, Paris (FR); Scott A. Small, Millerton, NY (US)

Correspondence Address: BAKER BOTTS L.L.P., 30 ROCKEFELLER PLAZA, 44TH FLOOR, NEW YORK, NY, 10112-4498, US

(21) Appl. No.: 12/077,612

(22) Filed: Mar. 19, 2008

Related U.S. Application Data
(63) Continuation of application No. PCT/US06/36460, filed on Sep. 19, 2006.
(60) Provisional application No. 60/718,582, filed on Sep. 19, 2005.

(51) Int. Cl. A61N 7/00

(52) U.S. Cl. 601/2

(57) ABSTRACT

A system and method for opening the blood-brain barrier in the brain of a subject is disclosed. In some embodiments, a region of the brain of a subject is targeted for opening; and a focused ultrasound beam is applied through the skull of the subject to the targeted region to open the blood-brain barrier in the brain of the subject.

### CROSS REFERENCE TO RELATED APPLICATIONS

This application is a continuation of PCT International Application No. PCT/US2006/0036460 filed Sep. 19, 2006 which claims the benefit of U.S. Provisional Patent Application Ser. No. 60/718,582, filed on Sep. 19, 2005, entitled “Methods and Apparatus for Localized Drug-Delivery in the Brain of a Subject Using Focused Ultrasound,” both of which applications are incorporated by reference in their entireties herein, and to both of which priority is claimed. The patent or application file contains at least one drawing executed in color. Copies of this patent or patent application publication with color drawing(s) will be provided by the Office upon request and payment of the necessary fee.

### BACKGROUND OF THE INVENTION

1. Field Of The Invention

The disclosed subject matter relates to a system and methods for treatment of the brain of a subject, and more particularly to opening the blood-brain barrier of a subject.

2. Background

Many neurologic disorders remain intractable to treatment by therapeutic agents because of the brain's natural defense, the blood-brain barrier (BBB). The BBB is a specialized vascular system consisting of endothelial cells with highly selective membranes connected together by tight junctions. This system impedes entry of virtually all large molecules from blood to brain tissue, rendering otherwise potent neurologically active substances and drugs ineffective simply because they cannot be delivered to where they are most needed. As a result, traversing the BBB is often a rate-limiting factor in brain drug delivery development

Many of the techniques currently being investigated to deliver drugs through the BBB have disadvantages. For example, the process of “lipidization” incorporates lipid groups to the polar ends of molecules to increase the permeability of the agent. While this technique increases the permeability of the drug in the targeted brain region, it does not have a localized effect and also increases permeability throughout the entire body. Consequently, drug dosage must be limited because of the risk of side effects. Another technique under study is neurosurgically-based drug delivery methods, where drugs are introduced into a region by a needle. The drug introduced by the needle spreads through diffusion and is typically localized to the targeted region. However, the diffusion mechanism does not allow for molecules to travel far from their point of release. In addition, the needle procedure invasively traverses untargeted brain tissue, potentially causing unnecessary damage. Other techniques utilize solvents mixed with drugs or adjuvants (pharmacologic agents) attached to drugs to disrupt the BBB through dilation and contraction of the blood vessels. However, this disruption is typically not localized within the brain, and the solvents and adjuvants used are potentially toxic. According to another technique, by studying the structure and function of transporters endogenous to the cell membrane of the endothelial cells, intense chemical modifications of drugs may allow their passage through these transporters. While this approach may provide a delivery technique specific to the brain, it requires special attention to each type of drug molecule and a specific transport system, resulting in a time consuming and costly process. Moreover, the drug transport may nevertheless not be completely localized to the targeted region.

Other techniques to open the BBB include use of focused ultrasound (FUS). However, several studies have shown that currently known FUS techniques, while locally disrupting the BBB, often also causes undesired tissue damage.

The introduction of microbubbles in conjunction with FUS has been found to open the BBB transiently. However, the approach is highly invasive and unacceptable for use with human subjects since it requires performing a craniotomy on the subject, replacing the skin, and allowing the wound to heal prior to sonication. In addition, the FUS fields were generated by a complicated array including a 16-sector transducer, in which each sector was driven with separate identical radio-frequency signals generated by a multichannel driving system. The multi-element transducers for multi-phasing are costly and difficult to manufacture.

All of these above-described techniques have inherent disadvantages. Accordingly, there is a need in the art for a system which can open the BBB barrier in a transient, localized and non-invasive manner, and which overcomes the limitations of the prior art.

### SUMMARY

Systems and methods for opening the blood-brain barrier in the brain of a subject are disclosed. In some embodiments, the systems and methods include targeting a region of the brain of a subject for opening; and applying an ultrasound beam through the skull of the subject to the targeted region to open the blood-brain barrier in the brain of the subject.

In some embodiments, applying an ultrasound beam through the skull of the subject may include generating an ultrasound beam by a single-element focused transducer. In some embodiments, applying an ultrasound beam through the skull of the subject may include generating an ultrasound beam by a plurality of single-element focused transducers. Applying a focused ultrasound beam through the skull of the subject may comprise generating a focused ultrasound beam having a frequency of about 1.525 MHz. Applying a focused ultrasound beam through the skull of the subject may comprise generating a focused ultrasound beam having an acoustic pressure at the focus of about 0.5 to 2.7 MPa.

In some embodiments, applying a focused ultrasound beam through the skull of the subject may include generating a focused ultrasound beam having a burst rate of about 10 Hz. Applying a focused ultrasound beam through the skull of the subject may comprise generating a focused ultrasound beam having a burst duration of about 20 ms. The ultrasound beam may include a plurality of shots having a duration and a delay between successive shots. The duration of the shots may be about 30 seconds. The delay between successive shots may be about 30 seconds.

In some embodiments, the method may further include administering a molecule to the subject for passage across the BBB. The molecule may include a drug, a contrast agent, or microbubbles into the bloodstream of the subject. Microbubbles may be filled with a drug or a contrast agent or a combination of the above. The focused ultrasound beam comprises a focus and applying a focused ultrasound beam through the skull of the subject may include generating a focused ultrasound beam having an acoustic pressure at the focus of about 0.8 MPa.

In some embodiments, targeting a region of the brain comprises targeting selected brain tissue. In some embodiment, targeting a region of the brain comprises targeting the vasculature surrounding selected brain tissue. Targeting may include locating at least one anatomical landmark of the subject. The method may further include positioning the focus of the ultrasound beam a predetermined distance from the anatomical landmark. Targeting a region of the brain may further comprise generating an image of the subject and the member. In some embodiments, targeting a region of the brain may comprise positioning the focus by reference to the image. A technique for targeting a region of the brain may further comprise positioning a member on adjacent said anatomical landmark, generating an image of the subject and the member, and positioning the focus by reference to the image.

Further features of the disclosed subject matter will be apparent from the accompanying drawings and the following detailed description of illustrative embodiments.

### BRIEF DESCRIPTION OF THE DRAWINGS

FIG. 1(a) is a diagram illustrating the system in accordance with an embodiment of the present subject matter.

FIG. 1(b) is a diagram illustrating the system in accordance with another embodiment of the present subject matter.

FIG. 2 illustrates a beam profile of an ultrasound beam in water in accordance with an embodiment of the present subject matter.

FIG. 3 illustrates a beam profile of an ultrasound beam through an ex vivo skull in accordance with an embodiment of the present subject matter.

FIG. 4 illustrates a top view of a subject's skill indicating anatomical landmarks.

FIGS. 5-6 illustrate a technique for targeting a portion of the brain of a subject by reference to the anatomical landmarks of the skull in accordance with an embodiment of the present subject matter.

FIG. 7 illustrates a histology cross section through the brain of a subject indicating the portion of the brain to be targeted in accordance with an embodiment of the present subject matter.

FIG. 8 illustrates a lateral 2-D raster scan of an apparatus for targeting a portion of the brain of a subject in accordance with an embodiment of the present subject matter.

FIG. 9(a) illustrates a T1 MRI scan of a horizontal slice of a subject brain obtained after sonication with a pressure amplitude of 2.0 MPa and 10 minutes after gadolinium injection in accordance with an embodiment of the present subject matter. Optison was injected 15 minutes before sonication.

FIG. 9(b) illustrates a T1 MRI scan of a horizontal slice of a subject brain obtained after sonication with a pressure amplitude of 2.0 MPa and 35 minutes after gadolinium injection in accordance with an embodiment of the present subject matter. Optison was injected 15 minutes before sonication.

FIG. 9(c) illustrates a T1 MRI scan of a horizontal slice of a subject brain obtained after sonication with a pressure amplitude of 2.0 MPa and 95 minutes after gadolinium injection in accordance with an embodiment of the present subject matter. Optison was injected 15 minutes before sonication.

FIG. 10(a) illustrates a T1 MRI scan of a horizontal slice of a subject brain obtained after sonication with a pressure amplitude of 2.5 MPa and 10 minutes after gadolinium injection in accordance with an embodiment of the present subject matter. Optison was injected 15 minutes before sonication.

FIG. 10(b) illustrates a T1 MRI scan of a horizontal slice of a subject brain obtained after sonication with a pressure amplitude of 2.5 MPa and 35 minutes after gadolinium injection in accordance with an embodiment of the present subject matter. Optison was injected 15 minutes before sonication.

FIG. 10(c) illustrates a T1 MRI scan of a horizontal slice of a subject brain obtained after sonication with a pressure amplitude of 2.5 MPa and 95 minutes after gadolinium injection in accordance with an embodiment of the present subject matter. Optison was injected 15 minutes before sonication.

FIG. 11(a) illustrates a T2 MRI scan of a horizontal slice of a subject brain obtained after sonication with a pressure amplitude of 2.5 MPa and 20 minutes after gadolinium injection in accordance with an embodiment of the present subject matter. Optison was injected 15 minutes before sonication.

FIG. 11(b) illustrates a T2 MRI scan of a horizontal slice of a subject brain obtained after sonication with a pressure amplitude of 2.5 MPa and 50 minutes after gadolinium injection in accordance with an embodiment of the present subject matter. Optison was injected 15 minutes before sonication.

FIG. 12(a) illustrates a T1 MRI scan of a horizontal slice of a subject brain obtained after sonication with a pressure amplitude of 2.7 MPa and 10 minutes after gadolinium injection in accordance with an embodiment of the present subject matter. Optison was injected 15 minutes before sonication.

FIG. 12(b) illustrates a T1 MRI scan of a horizontal slice of a subject brain obtained after sonication with a pressure amplitude of 2.7 MPa and 35 minutes after gadolinium injection in accordance with an embodiment of the present subject matter. Optison was injected 15 minutes before sonication.

FIG. 12(c) illustrates a T1 MRI scan of a horizontal slice of a subject brain obtained after sonication with a pressure amplitude of 2.7 MPa and 95 minutes after gadolinium injection in accordance with an embodiment of the present subject matter. Optison was injected 15 minutes before sonication.

FIG. 13(a) illustrates a T2 MRI scan of a horizontal slice of a subject brain obtained after sonication with a pressure amplitude of 2.7 MPa and 20 minutes after gadolinium injection in accordance with an embodiment of the present subject matter. Optison was injected 15 minutes before sonication.

FIG. 13(b) illustrates a T2 MRI scan of a horizontal slice of a subject brain obtained after sonication with a pressure amplitude of 2.7 MPa and 50 minutes after gadolinium injection in accordance with an embodiment of the present subject matter. Optison was injected 15 minutes before sonication.

FIG. 14 illustrates a T1 MRI scan of a horizontal slice of a subject brain obtained after sonication with a pressure amplitude of 0.8 MPa and 95 minutes after gadolinium injection in accordance with an embodiment of the present subject matter. Optison was injected 1 minute before sonication.

FIGS. 15(a)-(b) illustrate histologic sections using a crystal violet stain of the hippocampus, taken after sonication at 2.7 MPa without Optison injection.

FIG. 16 is a diagram illustrating the system in accordance with an embodiment of the present subject matter.

FIG. 17 illustrates a timeline in accordance with an embodiment of the present subject matter.

FIG. 18(a) illustrates a top view of a subject's skull indicating anatomical landmarks.

FIG. 18(b)-(c) illustrate cross-sections of a portion of the subject's brain.

FIGS. 19(a)-(h) illustrate MR images of a subject's brain after sonication in accordance with the present subject matter.

FIGS. 20(a)-(h) illustrate MR images of another subject's brain after sonication in accordance with the present subject matter.

FIGS. 21(a)-(h) illustrate MR images of a further subject's brain after sonication in accordance with the present subject matter.

FIGS. 22(a)-(h) illustrate MR images of yet another subject's brain after sonication in accordance with the present subject matter.

FIG. 23(a) is an MR image of a subject's brain in accordance with the present subject matter.

FIG. 23(b) illustrates a time plot of the BBB opening in according with the present subject matter.

FIGS. 24(a)-(b) illustrate spatio-temporal maps of a subject's brain after sonication in accordance with the present subject matter.

FIGS. 24(c)-(d) illustrate spatio-temporal maps of another subject's brain after sonication in accordance with the present subject matter.

FIGS. 24(e)-(f) illustrate spatio-temporal maps of a further subject's brain after sonication in accordance with the present subject matter.

FIGS. 25(a)-(b) illustrate spatio-temporal maps of a subject's brain after sonication in accordance with the present subject matter.

FIGS. 25(c)-(d) illustrate spatio-temporal maps of another subject's brain after sonication in accordance with the present subject matter.

FIGS. 25(e)-(f) illustrate spatio-temporal maps of a further subject's brain after sonication in accordance with the present subject matter.

FIG. 26(a) is an MR image of a subject's brain in accordance with the present subject matter.

FIGS. 26(b)-(i) are enlarged MR images of a subject's brain in accordance with the present subject matter.

FIG. 27 is a time plot illustrating the cross-sectional area of contrast enhancement after gadolinium injection in accordance with the present subject matter.

FIGS. 28(a)-(c) are histologic sections of a subject's brain in accordance with the present subject matter.

FIGS. 29(a)-(b) are histologic sections of a subject's brain in accordance with the present subject matter.

FIG. 30 is a fluorescence image of a subject's brain in accordance with the present subject matter.

FIG. 31(a) illustrates a representation of fluorescence intensity of the left hippocampus over the right hippocampus in accordance with the present subject matter.

FIG. 31(b) illustrates the ratio of fluorescence intensity in accordance with the present subject matter.

FIGS. 32(a)-(j) illustrate horizontal sections of a subject's brain in accordance with the present subject matter.

FIGS. 33(a)-(c) illustrate coronal sections of a subject's brain in accordance with the present subject matter.

### DETAILED DESCRIPTION OF EXEMPLARY EMBODIMENTS

A system and technique for providing an opening in the BBB of a subject using focused ultrasound is described herein. Although the biochemical processes that result in the opening of the BBB is not completely understood, the term “opening the BBB” shall be generally used herein to refer to an increased susceptibility of the BBB to passage of molecules therethrough.

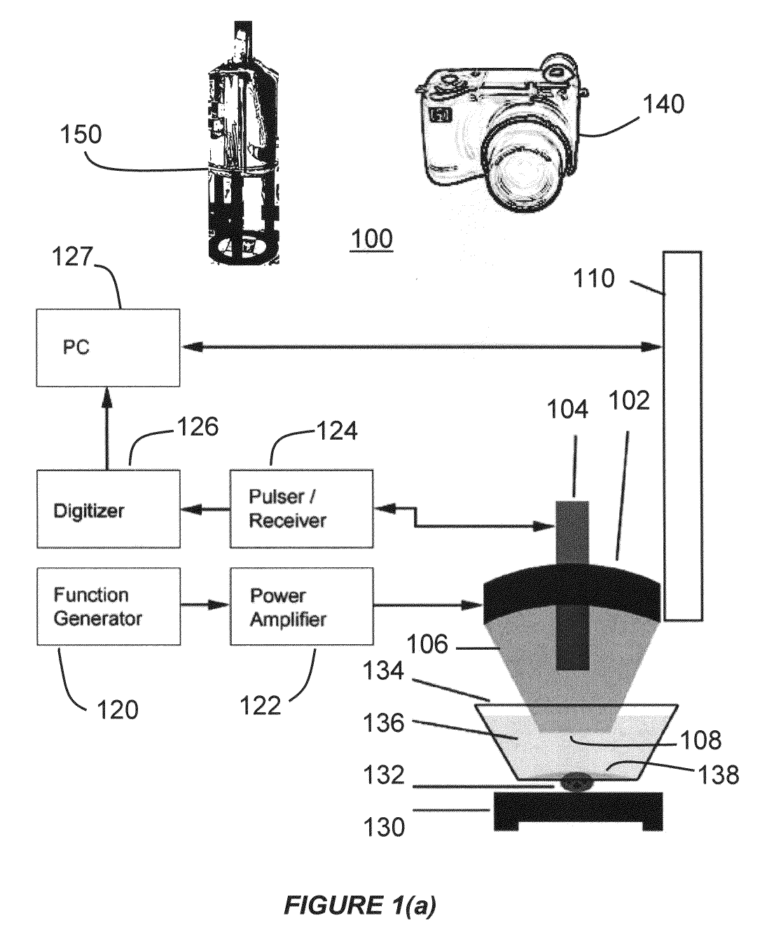

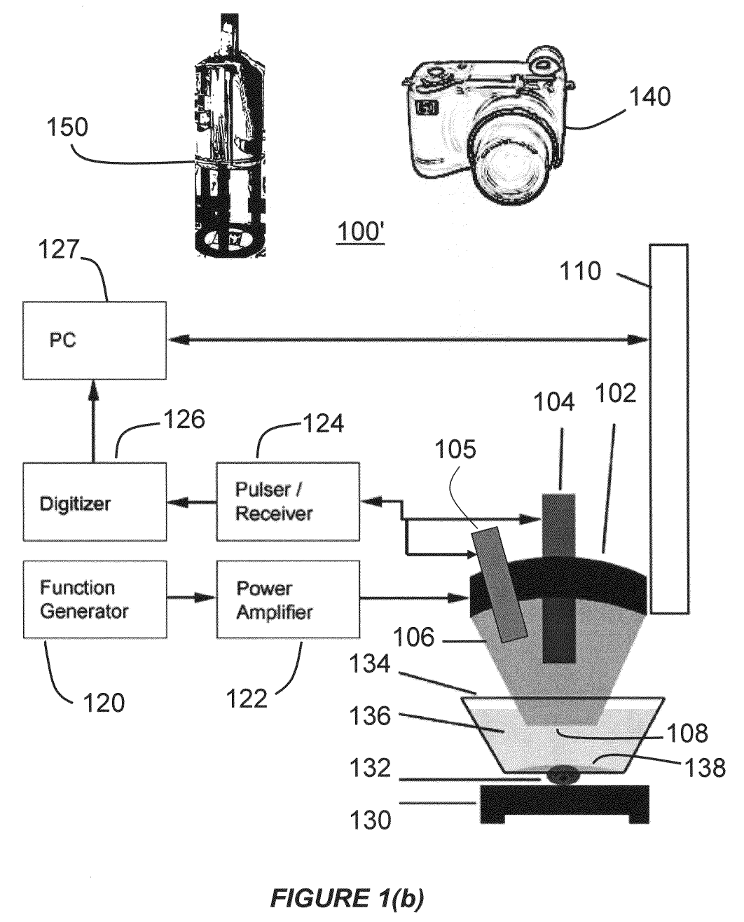

FIG. 1(a) illustrates an exemplary system for providing ultrasound waves, designated system 100. Ultrasound waves were generated by a FUS transducer, such as single-element circular-aperture FUS transducer 102. In the exemplary embodiment, FUS transducer 102 has a center frequency of 1.525 MHz, focal depth of 90 mm, an outer radius of 30 mm and an inner radius of 11.2 mm. FUS transducer 102 (Riverside Research Institute, NY) may be provided with a hole in its center for receipt of an imaging transducer, such as a single-element diagnostic transducer 104 (Riverside Research Institute, NY). In an exemplary embodiment, diagnostic transducer 104 has a center frequency of 7.5 MHz with a focal length of 60 mm. FUS transducer 102 and diagnostic transducer 104 may be positioned so that the foci of the two transducers are properly aligned. In the exemplary embodiment, a cone 106 filled with degassed and distilled water may be mounted on the transducer system 100. The cone may be manufactured from a clear plastic, such as polyurethane. The water may be contained in the cone 106 by capping it with a material considered substantially “transparent” to the ultrasound beam, such as a ultrathin polyurethane membrane 108 (Trojan; Church & Dwight Co., Inc., Princeton, N.J., USA).

The transducer assembly, which may include the FUS transducer 102 and the diagnostic transducer 104, may be mounted to a computer-controlled 3-D positioning system 110 (Velmex Inc., Lachine, QC. Canada), including motors VXM-1 and VXM-2 used in the exemplary embodiment. It is understood that other positioning systems may be incorporated for positioning the transducer assembly with respect to the targeted tissue.

In an exemplary embodiment, the FUS transducer 102 may be driven by a function generator 120, e.g., function generator HP33150A, manufactured by Agilent Technologies, Palo Alto, Calif., USA, through an amplifier 122, such as 50-dB power amplifier 3100L (ENI Inc., Rochester, N.Y., USA). The diagnostic transducer 104 may be driven by a pulser-receiver system 124, such as pulser-receiver 5052PR (Panametrics, Waltham, Mass., USA) connected to a digitizer 126, such as digitizer CS14200 (Gage Applied Technologies, Inc., Lachine, QC, Canada). It is understood that the above-described components may be modified or replaced with other components, as is known in the art, for producing the ultrasound beams described herein. PC 128 typically may include a processor, such a CPU (not shown), and may be any appropriate personal computer, or distributed computer system including a server and a client. For example, a computer useful for this system is Dell Precision 380 personal computer. It is understood that any personal computer, laptop, or other processor that can load software and communicate with the various components discussed herein may be used. A memory unit, such as a disk drive, flash memory, volatile memory, etc., may be used to store software for positioning and operating the transducer assembly, image data, a user interface software, and any other software which may be loaded onto the CPU.

In another embodiment, system 100′ may include a transducer assembly having an array of a plurality of single-element FUS transducers 104 and 105 which may be targeted to different regions of the brain of the subject. (FIG. 1(b)). Each FUS transducer 104, 105 in the array may be fired individually, thereby permitting opening of the BBB in several locations without repositioning the transducer assembly.

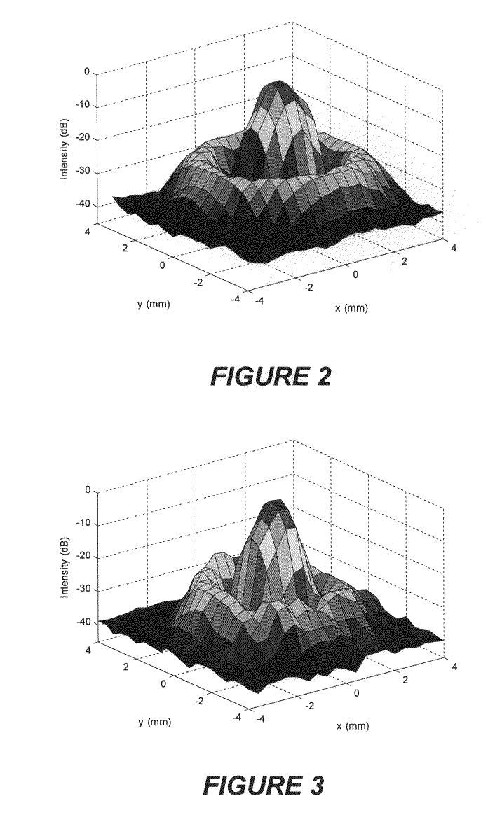

Prior to sonication and in order to verify undistorted propagation through the skull, a scan, such as a 3-D raster-scan (lateral step size: 0.2 mm; axial step size: 1.0 mm), of the beam of the FUS transducer 102, may optionally be performed in a large water tank containing degassed water with a needle hydrophone having a needle diameter on the order of about 0.2 mm (Precision Acoustics Ltd., Dorchester, Dorset, UK.) The dimensions of the beam provided by the FUS transmitter 102 may have a lateral and axial full-width at half-maximum (FWHM) intensity of approximately 1.32 and 13.0 mm, respectively, that in some embodiments may be approximately equal to the dimensions of the beam after propagation through the skull (see, e.g., FIG. 2).

System 100 also includes a platform for the subject. In the exemplary embodiment, the platform for the subject may be a polyurethane bed 130 for a smaller subject 132, such as a mouse. In this configuration, the membrane 138 may be placed over the subject 132. In other embodiments, the platform may be a hospital bed or surgical table, in which a larger subject (such as a human subject) may be laid prone or supine and the transducer assembly positioned on top of the region of the skull targeted.

Additional component(s) of the system 100 may include a targeting system for locating the focus of the FUS transducer 102 in the brain of the subject 132. In an exemplary embodiment, the targeting system may include a plurality of members, such as thin metal bars, e.g., 0.3 mm thin metal bars, fabricated from an acoustically reflective material, such as, e.g., paper clips. As will be described in greater detail below, the metal bars are placed on several landmarks of the skull of the subject to create a layout, or grid. Brain structures to be targeted, such as the hippocampus, are known to be located a particular distance from these landmarks. An image, such as a lateral 2-D raster scan, of the grid configuration is made using the diagnostic transducer 104. The location of the desired brain structure is identified relative to this grid. The focus of the FUS transducer may then be positioned to precisely target the desired brain structure. In another exemplary embodiment, the targeting system may include other imaging devices, such as a digital camera 140. For example, a digital camera may be used to photograph the head of the subject. The relevant landmarks may be identified in the photograph, and the focus of the FUS transducer targeted to a location relative to the landmarks. In addition, other MRI targeting equipment, as is known in the art, may be used for targeting the desired brain structure.

An exemplary method for delivering the molecule through the BBB is described herein. The subject is positioned on a platform. Subjects may be positioned in a prone position, and may be anesthetized for the sonication procedure. The degassed and distilled water bath 134 may be suspended over the subject's head. Ultrasound gel may be used to reduce any remaining impedance mismatches between the thin plastic layer 138 and the subject's skin. The transducer assembly may be placed in the water bath with its beam axis perpendicular to the surface of the skull.

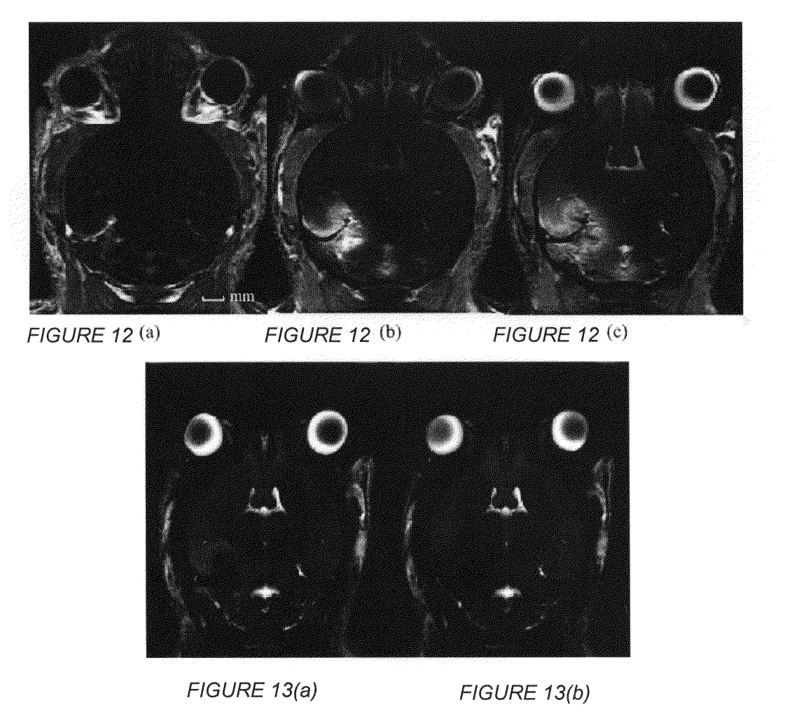

The focus of the transducer is positioned inside the subject's brain. The focus may be targeted to a region of the brain, such as the desired brain tissue, e.g., the hippocampus, or to the vasculature of the brain, e.g., arteries, ventricles, arterioles, and capillaries of the brain (see, e.g., FIG. 12(a) which targets the posterior cerebral artery). As discussed above, a targeting technique, such as the grid positioning method, may be used in an exemplary embodiment. In this method, the anatomic landmarks are used for targeting purposes. The location of the brain structure or vascular region were assumed relative to the landmarks based on known brain and skull anatomy. A grid consisting of thin metal bars may be placed in the water bath on top of the skull and in alignment with these landmarks. Using this grid positioning system, the brain structure may be reproducibly targeted when assumed to be at a location relative to the metal bars. An image, such as a lateral 2-D raster-scan, of the grid using the diagnostic transducer may be made and the location of the brain structure identified relative to this grid. The focus of the FUS transducer may then be placed in position by measuring distance with the diagnostic transducer. Targeting may also be performed by taking an image of the subject by photographic equipment, such as a digital camera.

The FUS transducer supplies the focused ultrasonic waves to the targeted area. For example, pulsed-wave FUS may be applied in a series of bursts having delays between bursts. In an exemplary embodiment, the burst rate is about 5 to 15 Hz, the burst duration is 20 ms, and the duty cycle is 20%. Exemplary acoustic pressures at the focus may be 0.5 to 3.0 MPa. According to this embodiment, the FUS was applied in a series of five shots lasting, e.g., 10-40 seconds each, with a delay between each shot of about 10-40 seconds. The FUS sonication procedure may be performed once or more on the subject's brain. The acoustic pressure values may be determined experimentally, for example, obtained from the values found in degassed water and corrected using the attenuation values of a skull similar to the subject's skull.

Following sonication, as described hereinabove, the BBB opens, thereby facilitating the passage of a molecule through the BBB. Such molecule may be a drug, medication or pharmaceutical compound, protein, antibody or biological material, chemical substance, contrast agent, or any other material to pass through the BBB. Such molecule may be administered to the subject by any known method. For example, the molecule may be injected into a vein of the subject. The molecule may administered intraperitoneally by a catheter. In some embodiments, the molecule may be administered orally. The administration of the molecule to the subject may occur prior to sonication, during sonication, or following sonication.

For example, an ultrasound contrast agent may be administered to the subject. Ultrasound scans of the subject may be used to determine whether the BBB has opened. A bolus of ultrasound contrast agent, e.g., Optison containing microbubbles, may be injected into a vein of the subject prior to sonication. In an exemplary embodiment, a 10 μL bolus (approximately 0.4 mL/kg) of Optison containing microbubbles having a mean diameter: 3.0 to 4.5 μm and a concentration of 5.0 to 8.0×108 bubbles per mL may be injected into the right femoral vein of the subject fifteen minutes prior to sonication. High-resolution echocardiogram equipment may be used following sonication to determine the presence of the ultrasound contrast agent. Microbubbles containing material such as a contrast agent or a drug may be administered to the subject for traversal of the BBB.

An MRI contrast agent may also be administered to the patient for passage through the BBB. MRI scans may be used to monitor opening of the BBB. In order to facilitate MRI scans during the procedure, an MRI system 150 maybe incorporated into the equipment described hereinabove. TI- and T2-weighted MRI scans may be obtained using a 1.5 T, 3.0 T, 9.4 T, or other, system (Bruker Medical; Boston, Mass. USA). For example, 0.5 mL of MRI contrast agent gadolinium (Omniscan; Amersham Health, AS Oslo, Norway) may be administered intraperitoneally via a catheter to depict BBB opening (Barzó et al. 1996). Intraperitoneal injection allows for the slow uptake of the MRI contrast agent into the bloodstream (Moreno et at 2006). After injection of the MRI contrast agent, a series of scans may be performed on the subject. For example, six alternating T1-weighted and T2-weighted fast spin-echo image scans, using the following specifications: a repetition time/echo time (TR/TE) of 4000 ms/9.2 ms; rapid acquisition with relaxation enhancement: 16; field of view (FOV) of 1.92×1.92 cm; matrix size of 256×256; number of slices: 10; slice thickness: 0.6 mm; slice gap: 0.1 mm; number of excitations (NEX): 10, 15 and 45.

Contrast-enhanced behavior may be followed for a period of time after injection of the contrast agent, to assess the time course of BBB opening. Detection of BBB opening may be detected by comparing an area of a nonsonicated homogeneous brain region with sonicated regions. Increased pixel intensity values of the sonicated regions which are increased above the values of the nonsonicated regions by a predetermined value, e.g., 2.5 standard deviations, are determined to be a contrast-enhanced region, revealing BBB opening. Higher resolution analysis may be used over an extended time period to determine the path of deposition of the molecule through the BBB.

### EXAMPLE A

### Ex Vivo FUS Administration in Mouse

The effect of the mouse skull on the ultrasound beam propagation using a single-element transducer was investigated with skulls of Brown CB57-b16 type mice (Charles River Laboratories, Wilmington, Mass., USA; mass: 23 to 28 g). The skull was excised and degassed in saline. Each skull was separately placed and held stationary in a tank filled with degassed water, such as the water bath discussed herein. The transducer assembly was submerged in the water tank and held stationary above the excised skull, with its focus placed 3 mm beneath the top of the skull. A needle hydrophone suspended from a computer-controlled 3-D positioning system was then placed at the beam focus. Two-dimensional lateral beam profiles at the focus without and with the skull were then measured. These measurements were made through several regions of the skull. Attenuation values were obtained by taking the difference between the pressure amplitude measured through the skull and the pressure amplitude measured in water and then dividing by the pressure amplitude in water. The mean attenuation value was finally obtained by averaging over the six attenuation values measured in six different skulls.

Ultrasound through the parietal bones on the left and right halves of the sagittal suture were found to provide the least amount of attenuation (˜18.1% of the pressure amplitude) when compared with other regions of the skull. The beam profile of the ultrasound beam at its focus in water only is illustrated in FIG. 2, and through an ex vivo skull in FIG. 3. The intensity values are in dB below the peak value. The full width at half maximum (FWHM) intensity was 1.33 mm through the skull and 1.32 mm when no skull was present. The change in the location of the focus as a result of skull aberration was lower than the resolution allowed by the needle hydrophone (needle diameter: 0.2 mm). The pressure field measurements revealed no significant distortion of the ultrasound beam shape or focus location.

### EXAMPLE B

### In Vivo FUS Administration in Mouse

Brown CB57-b16 type mice (Charles River Laboratories, Wilmington, Mass., USA; mass: 23 to 28 g) were used in sonication procedures. The mice were anesthetized with a mixture of ketamine (Fort Dodge Animal Health, Fort Dodge, Iowa, USA; concentration: 75 mg per kg of body mass) and xylazine (Ben Venue Laboratories, Bedford, Ohio, USA; concentration: 3.75 mg per kg of body mass). Before sonication, the hair on the top of the mouse heads was removed using an electric trimmer and a depilatory cream. After sonication, but before MRI scanning, the mice were switched to administration of isoflurane to simplify the long anesthesia procedure necessary for MRI scanning. During all imaging procedures, the vital signs of the mice were continuously monitored.

The mouse subject 132 was anesthetized and placed prone on the platform 130 (FIG. 1). A water bath 134, the bottom of which consisted of an ultrathin acoustically and optically transparent plastic layer 138, was filled with degassed and distilled water 136 and suspended over the anesthetized mouse's head 132. Ultrasound gel was used further to reduce any remaining impedance mismatches between the thin plastic layer 138 and the mouse skin. Finally, the FUS transducer 102 was placed in the water bath 134 with its beam axis perpendicular to the surface of the skull of the mouse subject 132.

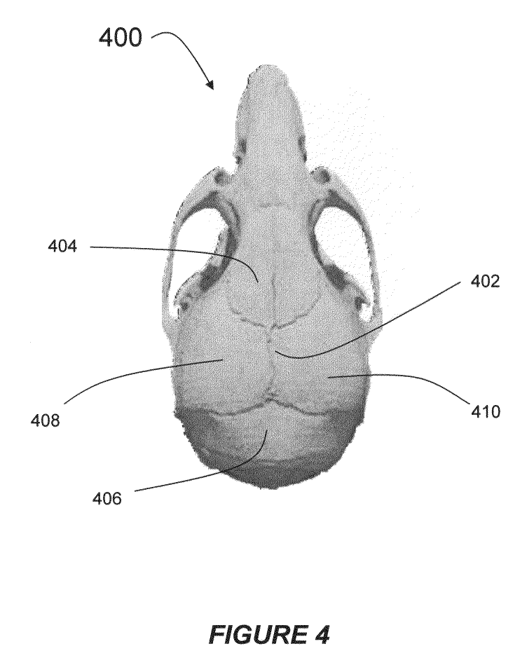

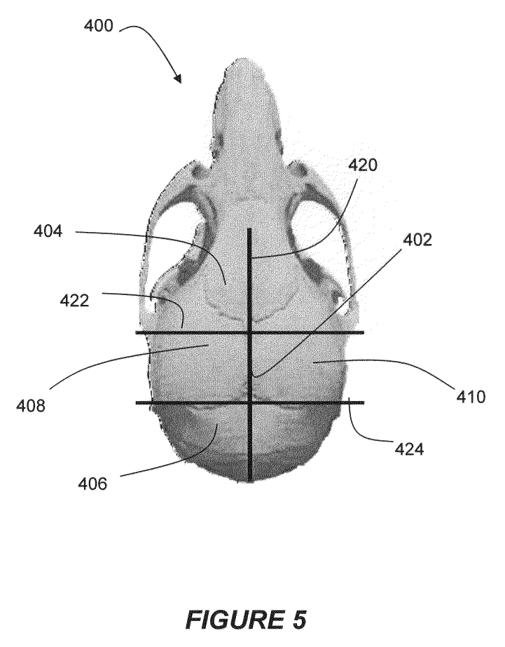

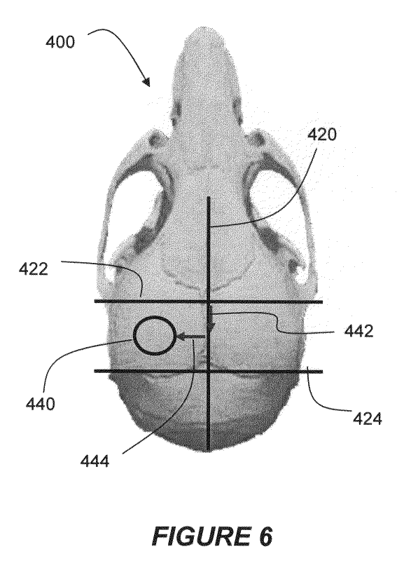

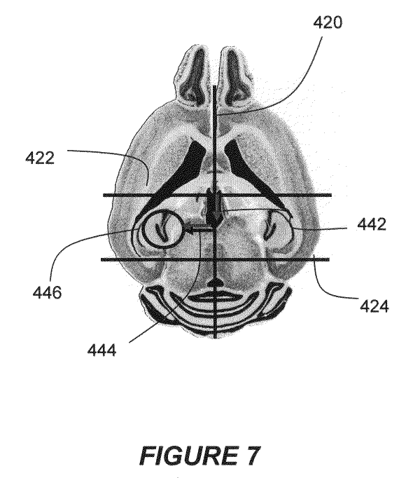

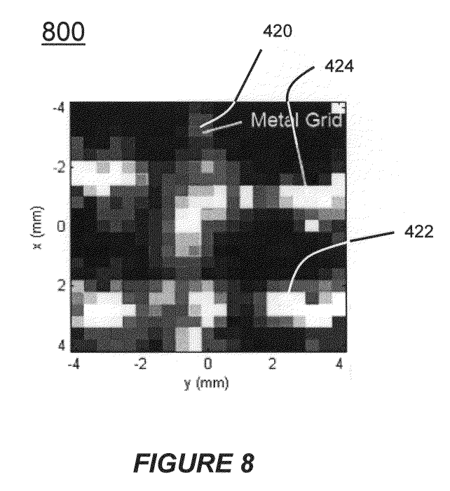

The focus of the transducer 102 was positioned inside the mouse brain using a grid positioning method, as discussed above. In this method, the sutures of the mouse skull seen through the skin were used as anatomic landmarks for targeting purposes. The location of the hippocampi were assumed relative to the sutures based on the mouse brain and known skull anatomy, as illustrated in FIG. 4. The landmarks of the mouse skull 400 include the sagittal suture 402, the frontal bone 404, the interparietal bone 406, the left parietal bone 408, and the right parietal bone 410. As illustrated in FIG. 5, a grid consisting of three equally spaced 0.3-mm thin F2 metal bars was placed in the water bath 134 on top of the skull 404 and in alignment with these sutures. The first bar 420 was aligned parallel and along the sagittal suture 402, and the second bar 424 was attached perpendicularly to the first bar and in alignment with the suture between the parietal and interparietal bone. In CB57-b16 type mice, these were the sutures that could be clearly seen through the skin. The third bar 422 was placed 4 mm away from and parallel to the second bar. Using this grid positioning system, FIG. 6 illustrates that the location of one of the hippocampi (indicated by circle 440) was reproducibly targeted when assumed to be at mid-distance (arrow 442) between the parallel bars and 2 mm away from the center bar (arrow 444). The actual location of the hippocampus 446 is indicated in the histology slice shown in FIG. 7. A lateral 2-D raster-scan 800 of the grid using the diagnostic transducer 104 was made and the location of the hippocampus was identified relative to this grid (FIG. 8). The focus of the FUS transducer 102 was placed 3 mm beneath the top of the skull by measuring distance with the diagnostic transducer 104. Using the grid positioning method and depth calculations, precise, accurate and reproducible targeting of the hippocampus of the mouse brain was performed.

To determine the accuracy of this positioning system, a separate set of preliminary experiments was performed. It was determined that the intended target was within 0.5 mm of the actual focus. Considering the 1.32-mm lateral FWHM beam used in these experiments, the grid positioning method was sufficiently precise to have the FUS beam consistently overlap the hippocampus of the murine brain. It is understood that more precise adjustments may be made as required by the subject and the particular organ or structure receiving sonication.

Ultrasound contrast medium was administered for transport across the BBB. A bolus of 10 μL (approximately 0.4 mL/kg) of ultrasound contrast agent (Optison) that contained microbubbles (mean diameter: 3.0 to 4.5 μm: concentration: 5.0 to 8.0×108 bubbles per mL) was injected into the right femoral vein of the mouse approximately 15 minutes before sonication (Table 1). Pulsed-wave FUS (burst rate: 10 Hz; burst duration: 20 ms; duty cycle: 20%; acoustic pressures at the focus: 2.0, 2.5 and 2.7 MPa) was then applied in a series of five shots lasting 30 seconds each with a 30 second delay between each shot. The FUS sonication procedure was performed once in each mouse brain. The acoustic pressure values were obtained from the values found in degassed water and corrected using the attenuation values of the skull, as discussed above. The sonications were focused at the left hippocampus of the mouse brain, and the right hippocampus was not targeted and acted as the control. The pressure values 2.0, 2.5 and 2.7 MPa were selected after a preliminary study that determined the threshold of BBB opening to be around 2.5 MPa, given the aforementioned set-up parameters.

TABLE 1

| Time (min.) | Procedure |
|---|---|
| 0 | Administration of anesthesia |
| 15 | Injection of Optison |
| 30 | Sonication |
| 45 | Start of T1 MRI scan acquisition (15 averages) |
| 60 | Injection of gadolinium |
| 60 | Start of T1 MRI scan acquisition (10 averages) |
| 70 | Start of T2 MRI scan acquisition (10 averages) |
| 80 | Start of T1 MRI scan acquisition (15 averages) |
| 95 | Start of T2 MRI scan acquisition (15 averages) |
| 110 | Start of T1 MRI scan acquisition (45 averages) |
| 155 | Start of T2 MRI scan acquisition (45 averages) |

To investigate the effect of the 15-minute delay between Optison injection and sonication, the presence of Optison in the bloodstream after such a time delay was verified. A separate study was performed, in which two mice were injected with Optison intravenously (IV) and their left ventricles were imaged with a high-resolution ultrasound system (Visual Sonics; Toronto, Ontario, Canada: frequency: 35 MHz). The echocardiograms indicated that Optison was still present at least 30 minutes after IV injection, and its concentration in the bloodstream was steadily decreasing over time. Although Optison was still present beyond 30 minutes, no further monitoring was performed. Finally, to further investigate the importance of the concentration of Optison (and, thus, timing of sonication post injection) on the BBB opening and the pressure amplitude used, the previously described sonication procedures were performed at 1-minute post injection of 25 μL of Optison at the lower pressure amplitude of 0.8 MPa.

The administration of an MRI contrast agent through the BBB was observed by use of TI- and T2-weighted MRI scans using a 9.4 T system (Bruker Medical; Boston, Mass. USA) (Table 1). The mice were placed in a plastic tube with a 3.8-cm diameter birdcage coil attached and were inserted vertically into the magnet. Approximately 15 minutes after sonication, but before MRI contrast agent injection, a TI-weighted spin-echo MRI scan was obtained (TRITE: 246.1 ms/10 ms; BW: 50,505.1 Hz; matrix size: 256×256; FOV: 1.92×1.92 cm; slice thickness: 0.6 mm: NEX: 10, 15 and 45). These images were useful to determine whether FUS had caused tissue damage. Once the first scan was completed, 0.5 mL of MRI contrast agent gadolinium (Omniscan; Amersham Health, AS Oslo, Norway) was administered intraperitoneally via a catheter to depict BBB opening. Intraperitoneal injection allowed for the slow uptake of the MRI contrast agent into the bloodstream. After injection of the MRI contrast agent, a series of six alternating T1-weighted and T2-weighted fast spin-echo image scans (TR/TE: 4000 ms/9.2 ms; rapid acquisition with relaxation enhancement: 16; FOV: 1.92×1.92 cm; matrix size: 256×256; number of slices: 10; slice thickness: 0.6 mm; slice gap: 0.1 mm; NEX: 10, 15 and 45) were performed after each mouse (Table 1).

Contrast-enhanced behavior was followed for a period of 140 minutes after injection of gadolinium, to assess the time course of BBB opening. For each MRI scan, a 15×15 pixel area of a nonsonicated homogeneous brain region was averaged. The entire MRI scan was then divided by this averaged value. The left (FUS-targeted) and right (control) hippocampi were compared in each mouse and any pixel intensity value above 2.5 standard deviations was determined to be a contrast-enhanced region, revealing BBB opening. Thresholding by 2.5 standard deviations was used because it provided a significantly clear differentiation between unaffected and BBB-opened regions in all mouse experiments. The approximate area of the BBB opening region was then calculated by counting the pixels above the threshold.

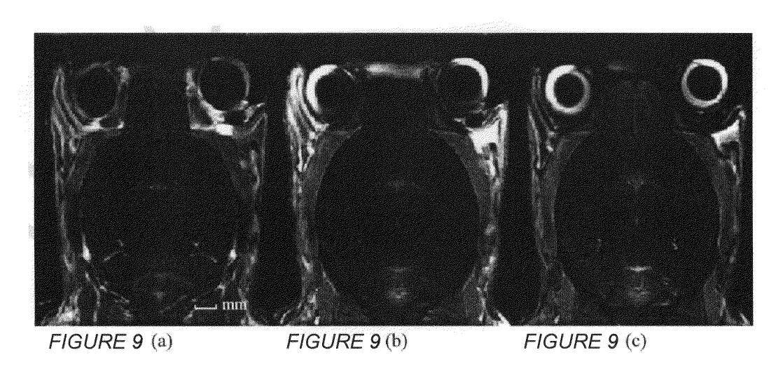

In most cases, no visible damage was detected on the T1 MRI scans obtained after sonication and before MRI contrast agent injection. Gadolinium was then injected to determine whether the BBB was opened. After sonication at a peak pressure amplitude of 2.0 MPa and injection with MRI contrast agent, no contrast enhancement was observed, as illustrated in FIGS. 9(a)-(c), which depict T1 MRI scans of horizontal slices of a single mouse brain approximately 3 mm beneath the top of the mouse skull. FIG. 9(a) was taken 10 minutes after gadolinium injection, FIG. 9(b) was taken 35 minutes after gadolinium injection, and FIG. 9(c) was taken 95 minutes after gadolinium injection. No contrast enhancement was discernible.

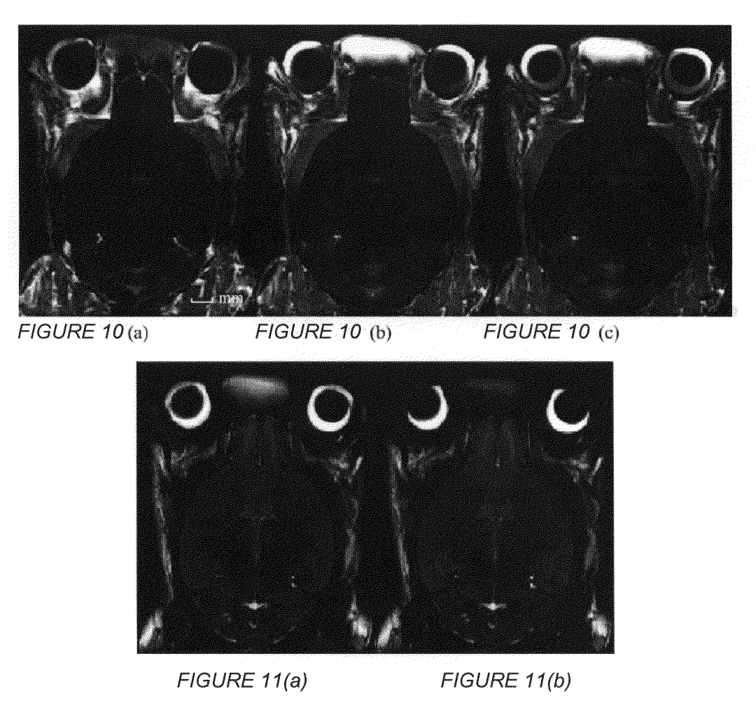

At 2.5 and 2.7 MPa, MRI contrast agent injection depicted BBB opening (FIGS. 10-13). A temporal analysis of this opening was made over a 140 minute period, revealing leakage of the MRI contrast agent from the posterior cerebral artery (PCA) or its adjacent arterioles and capillaries to the surrounding brain tissue. Sonication at a peak pressure amplitude of 2.5 MPa allowed for a highly localized opening of the BBB near the PCA, as illustrated in FIGS. 10(a)-(c) FIGS. 12(a)-(c) shows how, after sonication at a peak pressure amplitude of 2.7 MPa (as in the 2.5 MPa), the gadolinium first appears in the PCA only (10 min post injection, FIG. 12(a)), then slowly permeates throughout the surrounding regions (35 min post injection, FIG. 12(b)), eventually encompassing the entire left hippocampus (95 min post injection, FIG. 12(c)). On T2 MRI scans, BBB opening resulted in a decrease in pixel intensity and the area affected was in good agreement with what was seen on the TI MRI scans (see, FIGS. 11(a)-(b) and FIGS. 13(a)-(c)). Over the time studied, the area of contrast enhancement increased (Table 2).

TABLE 2

| Pressure | Area of contrast enhancement (mm²) | | |
|---|---|---|---|
| amplitude | At time = | At time = | At time = |
| (MPa) | 10 minutes | 10 minutes | 10 minutes |
| 2.0 | 0 | 0 | 0 |
| 2.5 | 0 | 8.40 | 33.1 |
| 2.7 | 33.5 | 116 | 152 |

Thus, at high resolution a detailed temporal and spatial analysis of the region enhanced by the MRI could be obtained, including accurate measurement of the region being affected by the BBB opening. For example, the vessel density and size appear to play a significant role in the way that the MRI contrast agent permeates the BBB. FIGS. 12(a)-(c) show how the MRI contrast agent first appears in the PCA or the region around the PCA (see, FIG. 12(a)) and then slowly permeates throughout the region (see, FIGS. 12(b) and 13(a)), eventually reaching the entire left hippocampus (see, FIGS. 12(c) and 13(b)). The method may thus be capable of determining the path of deposition of the molecule administered, e.g., contrast agent in this case, or of the drug release. In the exemplary study, the ultrasound focus encompassed an area greater than the PCA, but the initial dominant contrast enhancement appeared to occur in this region. At lower intensities (see, FIGS. 10 and 11), the opening of the BBB seems initially to be localized in the same blood vessel but only in the vessel branch that is parallel to the beam axis. The characteristic of drug delivery in the brain using this method will vary according to the vessel characteristics of the region where the focus of the ultrasound beam is positioned.

The extent of the region where BBB opening occurred also varied with ultrasound pressure amplitudes (see, FIGS. 10-13). At the pressure amplitude of 2.0 MPa, there was no opening of the BBB, and the area of BBB opening increased with the pressure amplitude above 2.5 MPa.

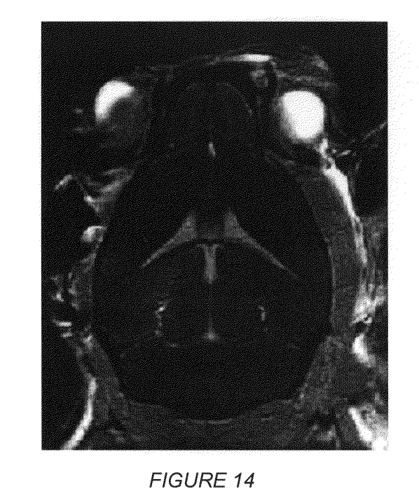

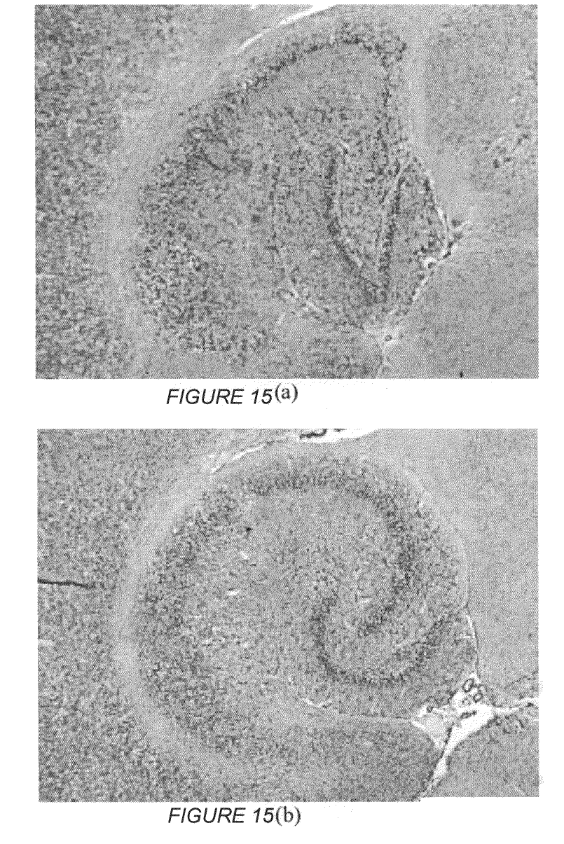

Finally, when the timing of Optison injection was changed from 15 minutes to 1 minute before FUS sonication and the injection was increased to 25 μL, opening of the BBB occurred at a far lower threshold of 0.8 MPa. FIG. 14 illustrates a T1 MRI scan obtained after sonication with a pressure amplitude of 0.8 MPa and 95 minutes after gadolinium injection. Option was injected 1 minute prior to sonication. Earlier injection and, thus, higher Optison concentration allowed for a reduced pressure amplitude necessary for BBB opening.

### EXAMPLE C

### In Vivo FUS Administration in Mouse

In an example, transgenic mice expressing mutant human APP_{K670N,M671L} protein (line Tg2576) and a line of mice expressing human mutant PS1_{M146V} protein (line 8.9) were crossed to generate offspring: doubly transgenic APP/PS1 animals and wild-type NTg littermates A total of six mice (age: 12 months, mass: 26-37 g) were used: transgenic APP/PS1 (n=3) and NTg (m=3) mice.

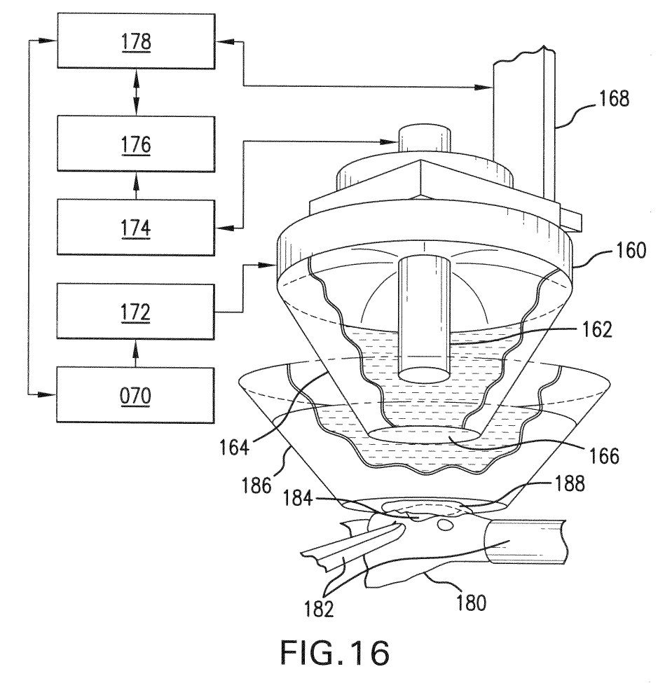

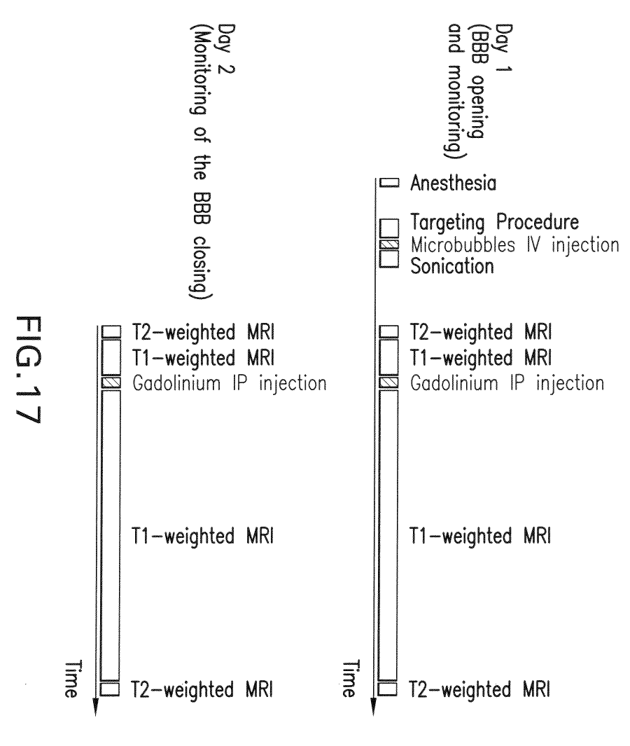

As illustrated in FIG. 16, ultrasound waves were generated by a single-element spherical segment FUS transducer 160 (center frequency: 1.525 MHz, focal depth: 90 mm, radius: 30 mm, Riverside Research Institute, New York, N.Y., USA). A pulse-echo diagnostic transducer 162 (center frequency: 7.5 MHz, focal length: 60 mm) was aligned through a central, circular hole (radius 11.2 mm) of the FUS transducer 160 so that the foci of the two transducers fully overlapped (FIG. 16). A cone 164 filled with degassed and distilled water was mounted onto the transducer system with the water contained in the cone by an acoustically transparent polyurethane membrane cap 166 (Trojan, Church & Dwight Co., Inc., Princeton, N.J., USA). The transducer system was attached to a computer-controlled, three-dimensional positioning system (Velmex Inc., Lachine, QC, CAN). The FUS transducer 160 was connected to a matching circuit and was driven by a computer-controlled function generator 170 (Agilent, Palo Alto, Calif., USA) and a 50-dB power amplifier 172 (ENI Inc., Rochester, N.Y., USA). The pulse-echo transducer was driven by a pulser-receiver system 174 (Panametrics, Waltham, Mass., USA) connected to a digitizer 176 (Gage Applied Technologies, Inc., Lachine, QC, CAN) in a personal computer 178 (PC, Dell Inc., TX, USA).

The pressure amplitudes and three-dimensional beam dimensions of the FUS transducer were measured using a needle hydrophone (Precision Acoustics Ltd., Dorchester, Dorset, UK, needle diameter: 0.2 mm) in a degassed water tank prior to the in vivo experiment. The pressure amplitudes reported in this paper were measured by calculating the difference between the peak-positive and peak-negative pressure values and attenuating by 18% to correct for skull attenuation. The lateral and axial full-width-at-half-maximum intensities of the beam were calculated to be approximately 1.32 and 13.0 mm, respectively.

The experimental timeline is depicted in FIG. 17. Each mouse 180 with intact skin and skull 182 was anesthetized using 1.25-2.50% isoflurane (SurgiVet, Smiths Medical PM, Inc., Wisconsin, USA) and placed prone with its head immobilized by a stereotaxic apparatus 184 (David Kopf Instruments, Tujunga, Calif.) that included a mouse head holder, ear bars, and a gas anesthesia mask (FIG. 16). The mouse hair was removed using an electric trimmer and a depilatory cream. A degassed water-filled container 186 sealed at the bottom with a thin, acoustically and optically transparent, Saran™ Wrap (Saran, SC Johnson, Racine, Wis., USA) was placed on top of the mouse head (FIG. 16). Ultrasound coupling gel 188 was also used to eliminate any remaining impedance mismatch. The FUS transducer was then submerged in the container with its beam axis perpendicular to the surface of the skull.

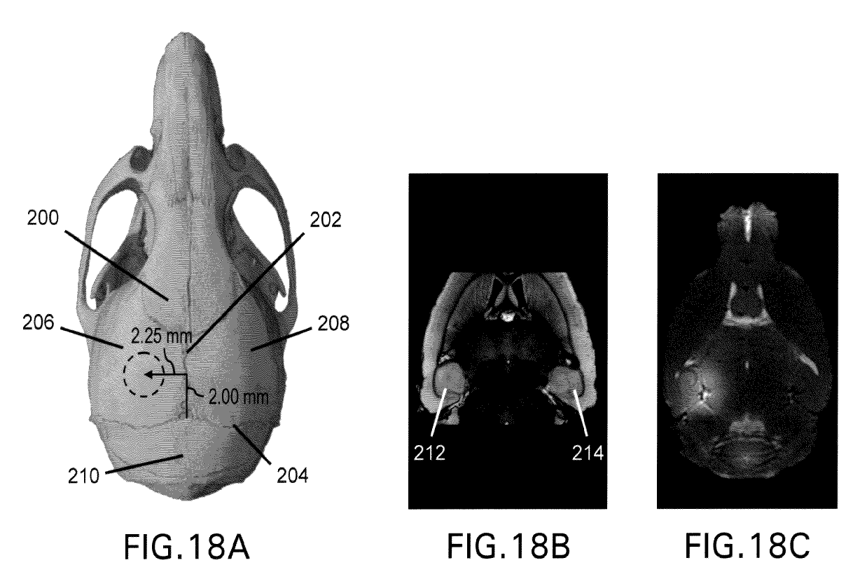

FIG. 18(A) illustrates the frontal bone 200, sagittal suture 202, lambdoid suture 204, left parietal bone 206, right parietal bone 208, and intraparietal bone 210. The focus of the transducer was positioned inside the mouse brain using a grid-positioning method. The beam axis of the transducer was aligned 2.25 mm away from the sagittal suture 202 and 2 mm away from the lambdoid suture 204 (FIG. 18(A)). The focal point was placed 3 mm beneath the top of the parietal bone of the skull (dotted line in FIG. 18(A)). In this placement, the focus of the FUS beam overlapped with the left hippocampus 212 and the left posterior cerebral artery (PCA) (FIG. 18(A)-(B)). The right hippocampus 214 was not targeted and was used as the control. After the BBB opening, gadolinium was injected and monitored with MRI (FIG. 18(C). The horizontal slices shown in FIGS. 18(B)-(C) were obtained approximately 3 mm beneath the top of the skull.

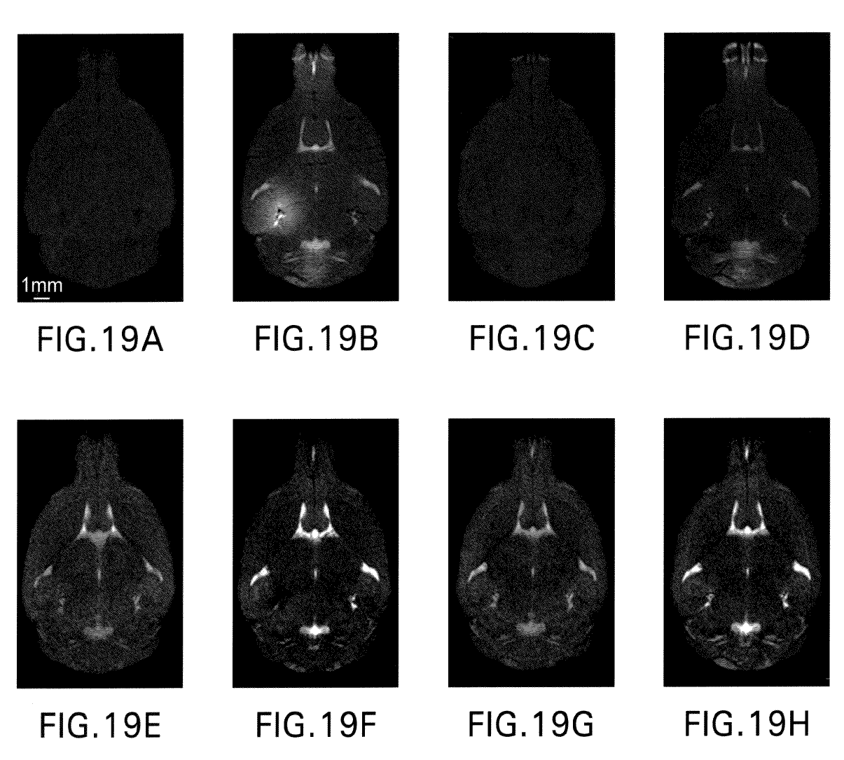

A bolus of 25 μl of ultrasound contrast agents (SonoVue®, Bracco Diagnostics, Inc., Milan, Italy) constituting of microbubbles (mean diameter: 3.0-4.5 μm, concentration: 5.0−8.0×10⁸ bubbles per ml) was injected into the tail vein 1-4 minutes prior to sonication (FIGS. 19-22) Pulsed FUS (pulse rate: 10 Hz, pulse duration: 20 ms, duty cycle: 20%) was then applied at 0.64 MPa peak-to-peak in a series of two shots consisting of 30 s of sonication at a single location (i.e., the hippocampus). Between each shot, a 30-s interval allowed for residual heat between pulses to dissipate. The FUS sonication procedure was performed once in each mouse brain.

A vertical bore 9.4-Tesla MR scanner (Bruker Medical, Boston, Mass., USA) was used to acquire images of mouse brains in two separate sessions. The first session consisted of a series of MRI scans on day 1, which were initiated 90 min after sonication to depict the BBB opening. The second session consisted of a series of MRI scans on day 2, which were initiated 22 hours after sonication to depict the BBB closure. In both sessions, the mice were immobilized in a plastic tube with a 3.8-cm-diameter birdcage coil attached and inserted vertically into the magnet. 1-2% isoflurane was administered while the respiration rate of the mouse was monitored throughout the entire MRI procedure. T₂-weighted (Repetition Time/Echo Time [TR/TE]: 4000 ms/9.2 ms, rapid acquisition with relaxation enhancement: 16, matrix size: 256×256, Field of View [FOV]: 1.92×2.14 cm, number of slices: 12, slice thickness: 0.6 mm, slice gap: 0.1 mm, Number of Excitations [NEX]: 5) and T₁-weighted (TR/TE: 246.1 ms/10 ms, Bandwidth: 50,505.1 Hz, matrix size: 256×256, FOV: 1.92×2.14 cm, number of slices: 12, slice thickness: 0.6 mm, slice gap: 0.1 mm, NEX: 5) MR sequences were both used. Pre-gadolinium T₂-weighted (n=1, duration: 4 min) and T₁-weighted MR images (n=3, duration: 12 min) were acquired to roughly determine whether FUS had caused any tissue damage and to check for any significant field inhomogeneities between the left and right hippocampi (FIG. 17). A gadolinium-based MRI contrast agent (Omniscan™, Amersham Health, AS Oslo, NOR, amount: 0.75 ml, molecular weight: 573.7 Da) that normally does not traverse the BBB was administered intraperitoneally (IP) via a catheter to depict the brain regions undergoing BBB opening and their spatio-temporal variation. IP injection allowed for the slow uptake of the MRI contrast agent into the bloodstream. Post-gadolinium sequential T₁-weighted (n=21, duration: 90 min) and T₂-weighted (n=1, duration: 4 min) MR images were then acquired. This MR imaging protocol was performed on day 1 (90 min after opening) and day 2 (22 hours after opening) to verify and map the extent of BBB opening and recovery, respectively. A relatively long delay between scans was required in order to allow for any residual IP-administered gadolinium to be cleared from circulation prior to the new injection.

FIGS. 19(a-h) are MR images of the first transgenic mouse brain after sonication at 0.64 MPa and injection of 25 μl of SonoVue®. T₁-weighted FIGS. 19(a-d) and T₂-weighted (FIGS. 19(e-h)) images were acquired 90 minuted post-sonication before (FIGS. 19(a and e)) and after (FIGS. 19(b and f)) gadolinium injection. Approximately 22 hours post-sonication, a second series of MR images were acquired before (FIGS. 19(c and g)) and 90 minutes after (FIGS. 19(d and h)) a second dosage of gadolinium injection.

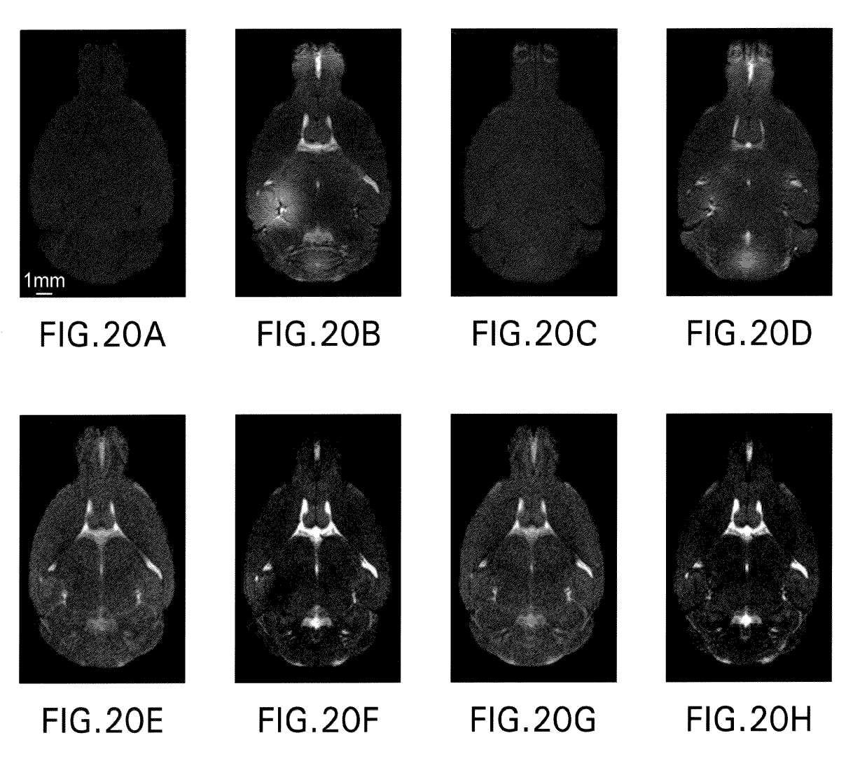

FIGS. 20(a-h) are MR images of the second transgenic mouse brain after sonication at 0.64 MPa and injection of 25 μl of SonoVue®(T₁-weighted FIGS. 20(a-d) and T₂-weighted (FIGS. 20(e-h)) images were acquired 90 minuted post-sonication before (FIGS. 20(a and e)) and after (FIGS. 20(b and f)) gadolinium injection. Approximately 22 hours post-sonication, a second series of MR images were acquired before (FIGS. 20(c and g)) and 90 minutes after (FIGS. 20(d and h)) a second dosage of gadolinium injection.

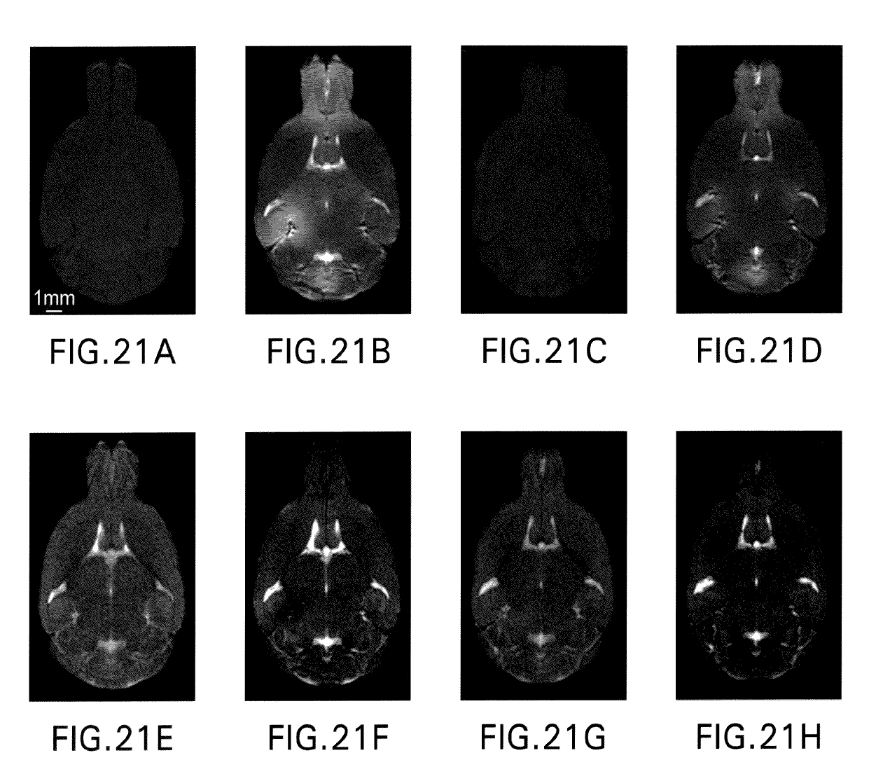

FIGS. 21(a-h) are MR images of the third transgenic mouse brain after sonication at 0.64 MPa and injection of 25 μl of SonoVue®. T₁-weighted FIGS. 21(a-d) and T₂-weighted (FIGS. 21(e-h)) images were acquired 90 minuted post-sonication before (FIGS. 21(a and e)) and after (FIGS. 21(b and f)) gadolinium injection. Approximately 22 hours post-sonication, a second series of MR images were acquired before (FIGS. 21 (c and g)) and 90 minutes after (FIGS. 21 (d and h)) a second dosage of gadolinium injection.

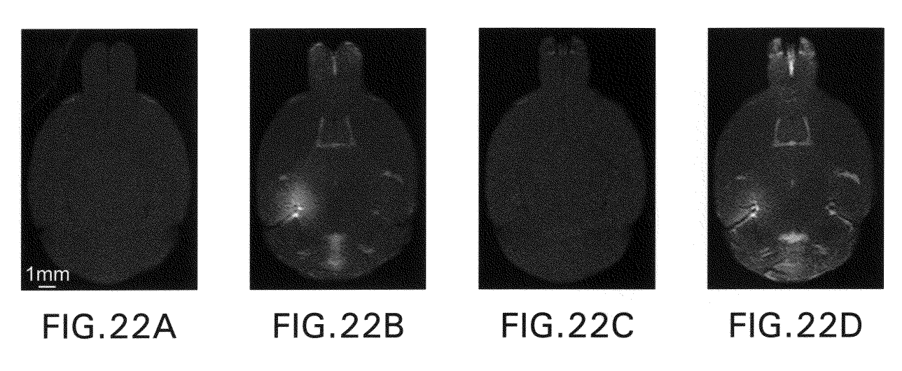

FIGS. 22(a-d) are MR images of a nontransgenic mouse brain after sonication at 0.64 MPa and injection of 25 μl of SonoVue®). T₁-weighted images (FIGS. 22(a-d)) were acquired 90 minuted post-sonication before (FIG. 22(a)) and after (FIG. 22(b)) gadolinium injection. Approximately 22 hours post-sonication, a second series of MR images were acquired before (FIG. 22(c)) and 90 minutes after (FIG. 22(d)) a second dosage of gadolinium injection.

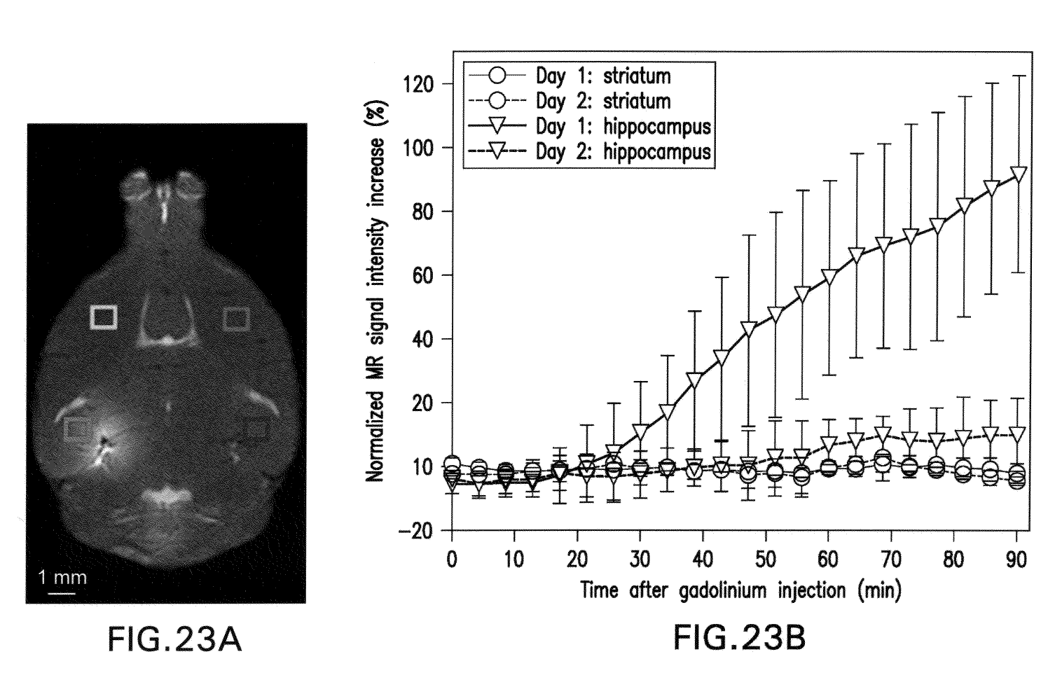

T₁-weighted images were processed with four objectives: (1) to identify the contrast enhanced pixels due to diffusion of gadolinium through BBB openings, (2) to quantify the mean signal intensity (SI) of the striatum and hippocampus regions of interest (ROI) over time, (3) to depict the area of contrast-enhancement in the hippocampus over time, and (4) to depict the spatial variation of the level of contrast-enhancement in the hippocampi at a single time point. In the first method, the mean SI of a 11×11 pixel (0.825×0.920 mm²) right hippocampus (control) ROI and its standard deviations (SD) were measured. The pixels of every image were each individually subtracted from this mean SI and any pixels above 2.5 SD were determined to be a contrast-enhanced pixel. A 2.5-SD threshold was used because it provided a statistically significant differentiation between intact and contrast-enhanced regions in all mouse experiments. In the second method, the mean SI of different ROIs were quantified over 90 min. The mean SI of a 11×11 pixel (0.825×0.920 mm²) ROI of the left and right striata and the left and right hippocampi were each separately measured (FIG. 23(A)). Quantitative analysis of the in vivo temporal changes in contrast enhancement was performed. Gadolinium deposition in the left (red) and right (blue) hippocampus, and the left (yellow) and right (green) striatum regions were monitored. The BBB in the left hippocampus was opened while the right hippocampus was not targeted and acted as the control. The striatum regions were not targeted. As illustrated in FIG. 23(B), the BBB opening after sonication at 0.62 MPa was monitored over time in the striatum (circles) and the hippocampus (triangles) regions. Contrast enhancement of the left hippocampus was observed on day 1 (solid) with less contrast enhancement observed on day 2 (dashed), indicating partial closing of the BBB.

The normalized increase in signal intensity (Sₙₒᵣₘ) for each region was then calculated:

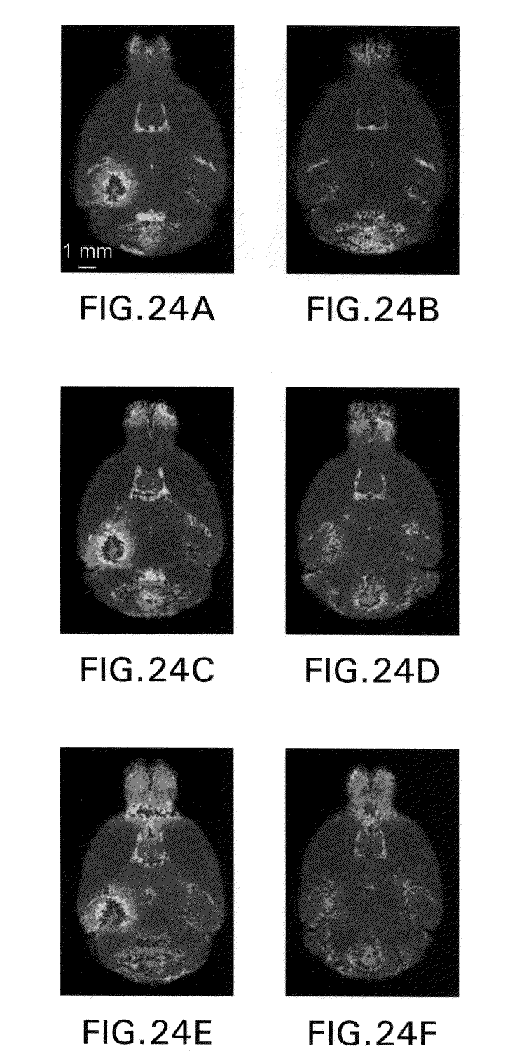

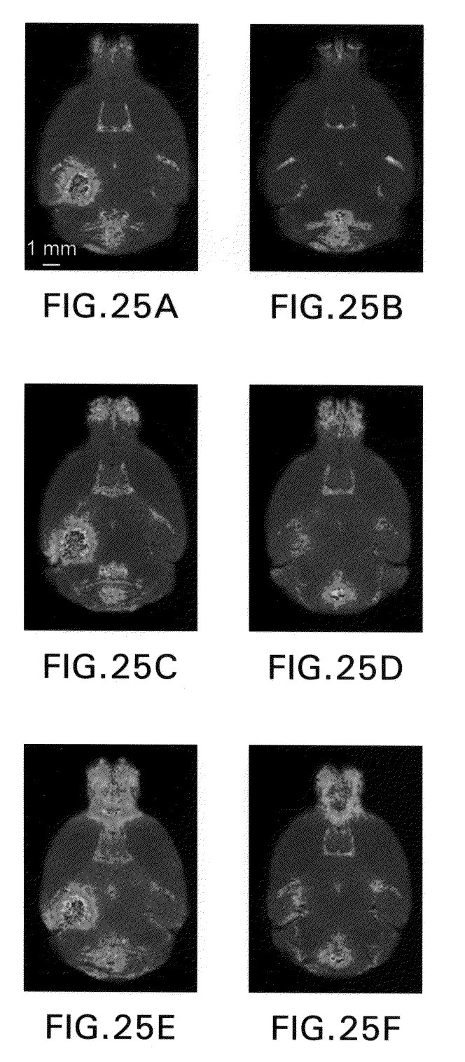

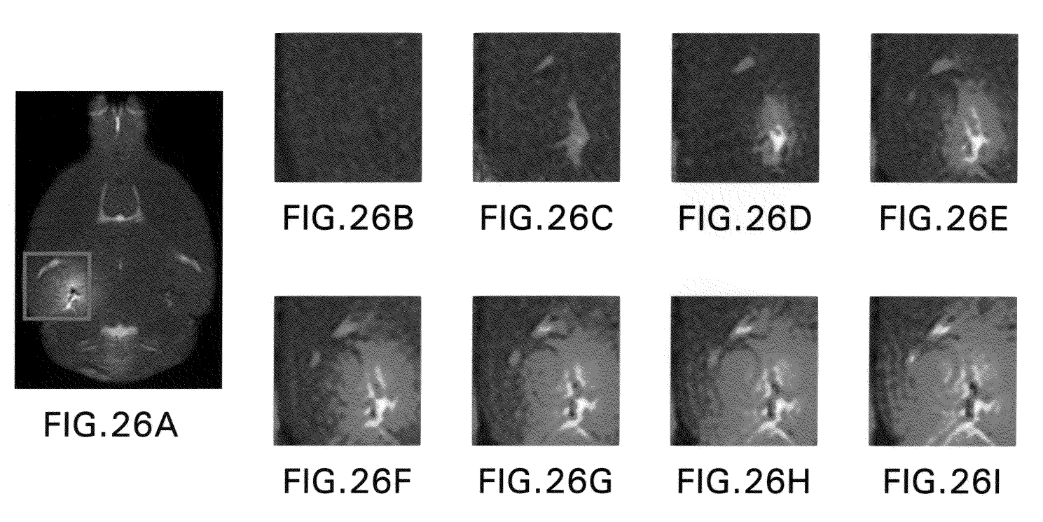

Sₙₒᵣₘ=100%×(SI_{left}×SI_{right})/SI_{right}

where SI_{left} and SI_{right} are the mean SI of the left and right ROIs. The normalized values from three independent mouse experiments were averaged, and the SDs were computed. In this manner, the percentage increase in SI of the left ROI compared to the contralateral right ROI was calculated. In the third method, time-varying spatial color maps depicting the temporal nature of contrast-enhancement were quantified. For each mouse, any SI above 2.5 SD of the mean SI of the right hippocampus ROI was determined to be a contrast-enhanced region. This was repeated at 13, 30, 47, 65, and 82 min post-gadolinium injection and pseudo-colored in red, blue, yellow, green, and cyan, respectively (FIG. 24). FIG. 24 illustrates in vivo spatio-temporal maps of the FUS-induced BBB opening 90 min (FIGS. 24(A, C, E)) and 22 hours (FIGS. 24(B, D, and F) after sonication at 0.64 MPa and injection of 25 μl of SonoVue® in three separate mice. The first (FIGS. 24(A and B)), second (FIGS. 24(C and D)), and third (FIGS. 24(E and F)) mouse brains were analyzed with sequential T₁-weighted images that depicted the slow diffusion of gadolinium 13 min (red), 30 min (blue), 47 min (yellow), 65 min (green), and 82 min (cyan) after its intreperitoneal injection. Using this thresholding technique, the area of contrast-enhancement was calculated over time elapsing after gadolinium injection (FIG. 26). FIG. 26(A) illustrates a magnified MR image of the BBB-opened left hippocampus (red box) using a black-red-white color map for better contrast. The MR images were acquired 0 minutes (FIG. 26(B)), 13 minutes (FIG. 26(C)), 25 minutes (FIG. 26(D)), 39 minutes (FIG. 26(E)), 51 minutes (FIG. 26(F)), 65 minutes (FIG. 26(G)), 78 minutes (FIG. 26(H)), and 90 minutes (FIG. 26(I)) post-gadolinium injection. Finally, spatially varying color maps depicting the levels of contrast-enhancement at a single time-point were calculated. For each mouse, the region of contrast-enhancement 2.5, 5.5, 8.5, 11.5, and 14.5 SDs above the mean SI of the right hippocampus ROI was separately color-coded in order to distinguish between areas of high and low depositions of gadolinium (FIG. 25). FIGS. 25(A-F) illustrate in vivo spatial maps of the FUS-induced BBB opening 90 min (as shown in FIGS. 25(A, C, and E)) and 22 hours (as shown in FIGS. 25(B, D, and F) after sonication at 0.64 MPa and injection of 25 μl of Sono Vue® in three separate mice. The first (FIGS. 25(A and B)), second (FIGS. 25(C and D), and third (FIGS. 25(E and F) mouse brains were analyzed 90 minutes post-gadolinium injection using T₁-weighted MRI. Regions of contrast enhancement above 2.5 (cyan), 5.5 (green), 8.5 (yellow), 11.5 (blue), and 14.5 (red) standard deviations above the mean signal intensity of the control (right) hippocampal region of interest were highlighted. The two color maps were finally overlaid onto the corresponding T₁-weighted image.

Approximately 1 hour after the final MRI session, each mouse was sacrificed and transcardially perfused with 30 ml of phosphate buffer saline (138 mM sodium chloride, 10 mM phosphate, pH 7.4), followed by 60 ml of 10% neutral buffered formalin. Its brain was removed, soaked in fixative for approximately 12 hours, embedded in paraffin, and horizontally sectioned with a sliding microtome. Sections at 6-μm thick were stained with hematoxylin and eosin (H&E) to evaluate macroscopic damage to the brain tissue while sections at 15-μm thick were stained with thioflavin S to confirm the existence of amyloid plaques.

MR images were acquired in the horizontal plane, as shown in the histology slice in FIG. 18B. Diffusion of gadolinium through the FUS-induced vessel openings in all transgenic APP/PS1 (FIGS. 19-21) and NTg (FIG. 22) mice enhanced the SI on the T₁-weighted (FIGS. 19B, 20B, 21B, and 22B) images while suppressing it on the T₂-weighted (FIGS. 19F, 20F, and 21F) images. On day 2, follow-up MRI scans of the same mice depicted significantly reduced T₁-weighted SI enhancement (FIGS. 19D, 20D, 21D, and 22D) and T₂-weighted SI suppression (FIGS. 19H, 20H, and 21H), indicating possible closure of the BBB. Quantitative temporal analyses of the contrast enhancement depicted a significant difference between the hippocampi and striata ROIs, and between the day 1 and day 2 hippocampi ROIs (FIG. 23). However, a few pixels in the targeted region exhibited contrast changes on day 2 (FIGS. 19D and F, 20D and F, 21D and F, and 22D).

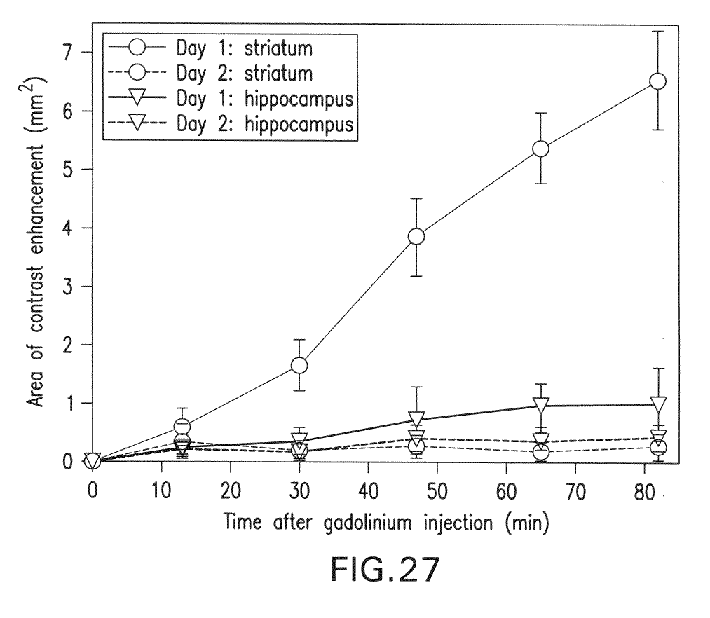

The deposition of gadolinium through the BBB openings varied spatially and temporally following its IP injection. Quantitatively, this was observed as an increase in the cross-sectional area of contrast enhancement over time (FIG. 27). FIG. 27 illustrates a cross-sectional area of contrast enhancement of the left (targeted) and right (control) hippocampi after IP gadolinium injection in the transgenic APP/PS1 mice. The data was extracted from two MRI scans obtained 90 minutes (illustrated with a circle) and 22 hours (illustrated with a triangle) after FUS-induced BBB opening. Using color maps, it was found that the site of initial contrast enhancement was at or near the PCA (FIGS. 24A, C, and E). Over time, the gadolinium gradually permeated into the surrounding regions until the entire hippocampus was contrast enhanced. Although gadolinium was successfully deposited throughout the targeted region at this final time point (90 min post-gadolinium injection), it should be noted that the degree of contrast enhancement was not spatially uniform. Regions of local minima and maxima of contrast enhancement were located throughout the targeted region of each mouse, i.e., there were voxels near the center of the targeted region which were not contrast enhanced (FIGS. 25A, C, and E). This indicates a high localization of gadolinium to specific regions throughout the hippocampus. The temporal progression of contrast enhancement is more clearly depicted in FIG. 26. On the following day, no contrast enhancement in most of the targeted region was observed, most notably in mouse 1 (FIGS. 24B and 25B). However, the targeted region of mouse 2 and 3 contained contrast enhanced pixels, especially near the large vessels (i.e., PCA region) (FIGS. 24D, F, 25D, and F). Regardless of these observations, the area of contrast enhancement (FIG. 27) and the degree of contrast enhancement (FIGS. 25B, D, and F) on day 2 were significantly reduced.

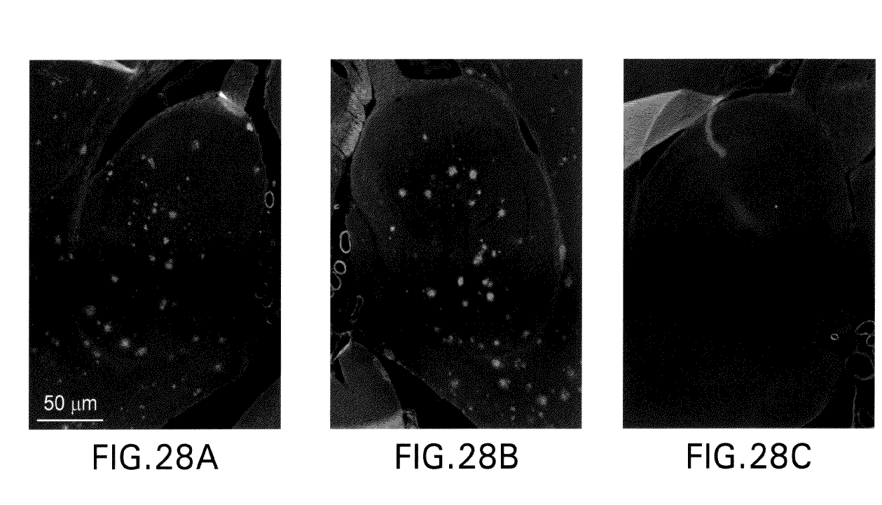

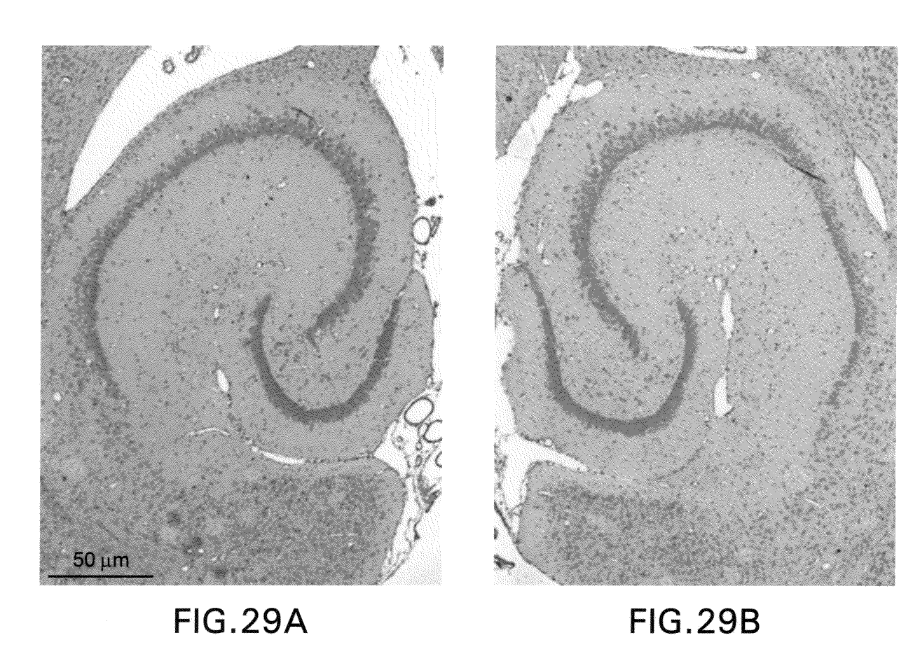

The existence of amyloid plaques in the transgenic APP/PS1 mice were confirmed with Thioflavin-S staining and fluorescence microscopy of the left (FIG. 28(A)) and right (FIG. 28(B)) hippocampi and compared with the left hippocampus of nontransgenic (control) mice (FIG. 28(C)). (The dark, diffuse band across each image is an artifact due to stitching several images together. There were no significant differences in the number of plaques on the left and right hippocampi. No macroscopic damage to the hippocampi was observed between the left and right hippocampi of the hematoxylin and eosin (“H&E”) stained histological sections of the left (FIG. 29(A)) and right (FIG. 29(B)) hippocampi of a transgenic APP/PS1 mouse.

A normally BBB-impermeable molecule was delivered through the left hippocampal vasculature of one-year-old transgenic APP/PS1 mice. The hippocampus was targeted, because it is the site initially and most significantly affected by AD. All sonications were applied in vivo, after microbubble injection, and through the intact skin and skull of the mouse using a single sonication generated by a single-element transducer.

BBB opening was monitored in vivo with a 9.4 T MRI scanner in all mice before and after gadolinium was injected. MR images were acquired on day 1 (90 min post-sonication) to monitor the BBB opening and day 2 (22 hours post-sonication) to monitor the BBB recovery. No significant contrast changes of the MRI SI were observed in pre-gadolinium T₁ and T₂-weighted images (FIGS. 19-21, A, C, E, and G, FIGS. 22A and C), indicating no significant field inhomogeneities and no residual gadolinium. Thus, the venous system had slowly cleared the residual gadolinium before the day 2 scan was initiated. After gadolinium was injected, the mean SI of the targeted left hippocampus ROI gradually depicted contrast-enhancement relative to the mean SI of the right (control) hippocampus ROI (FIG. 23). Eventually (90 min post-gadolinium injection), the entire left hippocampus and some of its surrounding regions were contrast-enhanced (FIGS. 19-21, ABEF, FIGS. 22A and B). As qualitatively indicated by the MR images, T₁-weighted images were more sensitive to contrast changes in the hippocampal region than T₂-weighted images while, at the same time, enhancing the anatomical structures within the targeted region.

The left hippocampal region was reliably and accurately targeted through the intact skin and skull in all transgenic APP/PS1 and control mice using a previously developed grid positioning system. A single sonication focus using a single-element FUS transducer was sufficient to repeatedly and reliably deposit the normally impermeable Omniscan™ into the left hippocampus and its surrounding regions (FIGS. 19-22). FUS-induced BBB opening has previously been indicated through contrast-enhancement of the MRI signal intensity. However, it should be noted that a contrast-enhanced pixel could be due to gadolinium traversing openings in the vasculature, diffusing through the interstitial tissue originating from BBB-opened vessels, or localization of gadolinium to certain regions of the brain or vasculature. Intraperitoneal injection, as opposed to IV injection, of gadolinium begins with transportation across the peritoneal microvasculature prior to venous transport. Its use allows for a larger amount of injectable gadolinium (0.75 ml), a long temporal analysis of the BBB opening (over 90 min), and a reduction in the mouse mortality and morbidity rates. The 90-min MRI scan time was previously determined to be sufficiently long for Omniscan™ to diffuse throughout the hippocampal parenchyma.

Color maps of both the temporal and spatial distributions of gadolinium were used in order to facilitate the visual interpretation of the MR images in the transgenic APP/PS1 mice (FIGS. 24 and 25). FIGS. 24A, C, and D depict the BBB opening 90 min post-sonication. Approximately 13-min post-gadolinium injection (red region), the contrast-enhanced region extended from the anterior to the posterior regions of the brain, and did not have the characteristic circular shape of the FUS-beam lateral cross-section. The dimensions and beam characteristics of the FUS transducer used is described in detail in our previous study. This lack of spatial uniformity or symmetry around the focal spot was observed, although to a lesser extent, with increasing time. In order to correctly label the vessels within the BBB opened regions, the vessels in the MR images were roughly matched to μCT vascular maps of similar mice. According to the maps, blood circulates from the PCA to the longitudinal and transverse hippocampal arteries, which supply the hippocampus with nutrients. These vessels are within the FUS-targeted region (a 1.3-mm-diameter cross-section). Since the MR images were obtained in the horizontal orientation, lateral cross-sections of the PCA or longitudinal hippocampal arteries are visible with transverse hippocampal arteries assumed to extend laterally clockwise from them and into the hippocampus. Due to the nature of the of the IP injection, contrast enhancement was first detected at the PCA or longitudinal hippocampal artery, and later in the transverse hippocampal arteries, or the region surrounding the hippocampal arteries (FIG. 26). At the final time-point (90 min post-gadolinium injection), local minima and maxima in the level of contrast enhancement were observed throughout the sonicated region (FIGS. 25A, C, and E). Gadolinium was highly concentrated in sub-millimeter sites (red regions). These localized high concentrations of gadolinium were not contiguous along a certain path but were instead dispersed throughout the entire targeted region.

Partial or nearly complete BBB closure was observed in all mice (FIG. 25 B,D, and F, 7D). BBB closing was observed in almost the entire targeted region in mouse 1 (FIG. 25B) while mouse 3 (FIG. 25F) had the longest time to closure. Regardless, the recoveries in all mice were significant with a low level of contrast enhancement on day 2 images (FIG. 23). As with the opening, the BBB closed at different degrees and at different locations throughout the targeted location.

There were no significant differences in the BBB opening and closing behaviors between the transgenic (APP/PS1) and NTg mice. No detectable differences in the timing and characteristics of the BBB opening behavior were found in the AD mice compared to that of the control mice. However, the BBB recovery appeared to be less complete in the NTg mice than the transgenic APP/PS1 mice (FIG. 22D). This may be due to several reasons. A total of two SonoVue® vials were used multiple times on different days for all six mice. Repeated use of vials may alter the properties of the microbubbles (i.e., bubble radius) due to the leakage of air into the vial from repeated injections to aspirate the mixture. Also, the weight of the mice varied and the accuracy of the amount of microbubbles injected (25 μl) was limited by the syringe used. All of these factors contributed to the variability between the transgenic (APP/PS1) and NTg mice. The overall behaviors of the two groups of mice were similar based on our measurements.

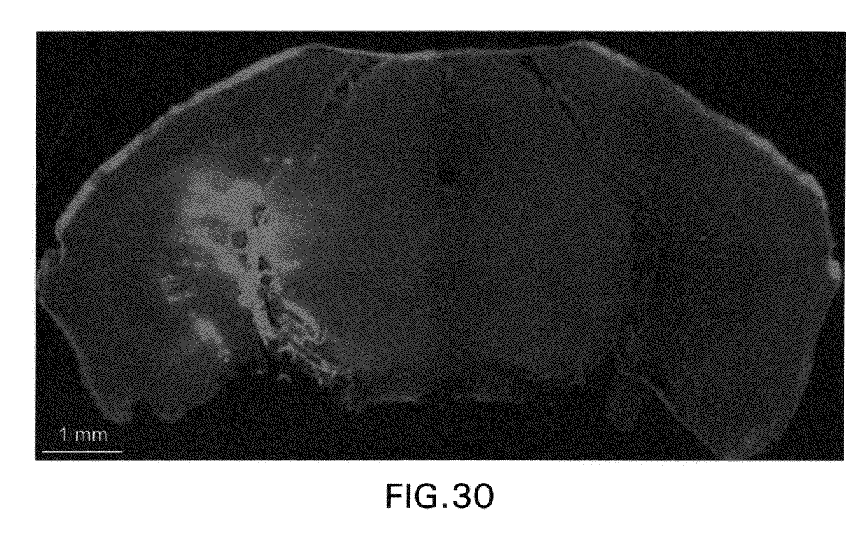

In its normal state, the BBB excludes more than 99% of all molecules greater than 400 Da. However, most neurologically potent molecules are greater than 400 Da. Two potential categories of drugs with disease-modifying behavior that do not traverse the BBB are β-secretase inhibitors and antibodies. β-secretase inhibitors are approximately 1000 Da in molecular weight while antibodies range from 30,000 to 200,000 Da. In this example, a 573.3 Da molecule, and, in a separate study, 3,000, 70,000 (FIG. 30), and 2,000,000 Da fluorescent dextran molecules were delivered trans-BBB. (FIG. 30 illustrates a fluorescence image of a nontransgenic mouse after FUS-induced BBB opening and injection of a 70,000 Da dextran molecule tagged with Texas Red® dye.) These molecules are far larger than most antibodies, and, therefore, the size of BBB opening is not a significant issue. Using the high resolution MR images acquired in a high magnetic field (9.4 T), it was observed that the BBB recovery varied temporally at different locations of the targeted region. Also, there were variations in the extent of BBB recovery between different mice. The ultrasound-induced BBB opening was observed to be transient in nature. In addition, the BBB opening in AD mice was found to be insignificantly different from control mice.

### EXAMPLE D

### In Vivo FUS Administration in Mouse

According to another example, ultrasound waves were generated by a single-element spherical segment FUS transducer (center frequency: 1.525 MHz, focal depth: 90 mm, radius: 30 mm, Riverside Research Institute, New York, N.Y., USA). A pulse-echo diagnostic transducer (center frequency: 7.5 MHz, focal length: 60 mm) was aligned through a central, circular hole (radius 11.2 mm) of the FUS transducer so that the foci of the two transducers fully overlapped (FIG. 1). A cone filled with degassed and distilled water was mounted onto the transducer system with the water contained in the cone by an acoustically transparent polyurethane membrane cap (Trojan, Church & Dwight Co., Inc., Princeton, N.J., USA). The transducer system was attached to a computer-controlled, three-dimensional positioning system (Velmex Inc., Lachine, QC, CAN). The FUS transducer was connected to a matching circuit and was driven by a computer-controlled function generator (Agilent, Palo Alto, Calif., USA) and a 50-dB power amplifier (ENI Inc., Rochester, N.Y., USA). The pulse-echo transducer was driven by a pulser-receiver system (Panametrics, Waltham, Mass., USA) connected to a digitizer (Gage Applied Technologies, Inc., Lachine, QC, CAN) in a personal computer (PC, Dell Inc., TX, USA).

The pressure amplitudes and three-dimensional beam dimensions of the FUS transducer were measured using a needle hydrophone (Precision Acoustics Ltd., Dorchester, Dorset, UK, needle diameter: 0.2 mm) in a degassed water tank prior to the in vivo procedures. The pressure amplitudes reported in this paper were measured by calculating the difference between the peak-positive and peak-negative pressure values and attenuating by 18% to correct for skull attenuation. The lateral and axial full-width-at-half-maximum intensities of the beam were calculated to be approximately 1.32 and 13.0 mm, respectively.

A total of fourteen wild-type mice (Harlan, Indianapolis, Ind., USA, strain: C57BL/6, mass: 28 to 32 g, sex: male) were used in this study. The mice were anesthetized using 1.25-2.50% isoflurane (SurgiVet, Smiths Medical PM, Inc., Wisconsin, USA) throughout both the BBB opening and transcardial perfusion procedures. Between the two procedures, the mice were laid underneath a heating lamp to prevent any the onset of hypothermia possible due to extended anesthesia.

Each mouse was anesthetized using 1.25-2.50% isoflurane (SurgiVet, Smiths Medical PM, Inc., Wisconsin, USA) and placed prone with its head immobilized by a stereotaxic apparatus (David Kopf Instruments, Tujunga, Calif.) that included a mouse head holder, ear bars, and a gas anesthesia mask (FIG. 16). The mouse hair was removed using an electric trimmer and a depilatory cream. A degassed water-filled container sealed at the bottom with a thin, acoustically and optically transparent, Saran™ Wrap (Saran, SC Johnson, Racine, Wis., USA) was placed on top of the mouse head (FIG. 16). Ultrasound coupling gel was also used to eliminate any remaining impedance mismatch. The FUS transducer was then submerged in the container with its beam axis perpendicular to the surface of the skull.

The focus of the transducer was positioned inside the mouse brain using a grid-positioning method. The beam axis of the transducer was aligned 2.25 mm away from the sagittal suture and 2 mm away from the lambdoid suture (FIG. 18A). The focal point was placed 3 mm beneath the top of the parietal bone of the skull. In this placement, the focus of the FUS beam overlapped with the left hippocampus and the left PCA (FIG. 18A-B). The right hippocampus was not targeted and was used as the control.

A 25 μl bolus of ultrasound contrast agents (SonoVue®, Bracco Diagnostics, Inc., Milan, Italy) constituting of microbubbles (mean diameter: 3.0-4.5 μm, concentration: 5.0−8.0×10⁸ bubbles per ml) was injected into the tail vein 1-4 minutes prior to sonication. Pulsed FUS (pulse rate: 10 Hz, pulse duration: 20 ms, duty cycle: 20%) was then applied at 0.64 MPa peak-to-peak in a series of two shots consisting of 30 s of sonication at a single location (i.e., the hippocampus). Between each shot, a 30-s interval allowed for residual heat between pulses to dissipate. The FUS sonication procedure was performed once in each mouse brain.

Following BBB opening, the fourteen mice were studied using five different dextran experimental parameters. Approximately 10 min after FUS-induced BBB opening, mice were injected with fluorescent-tagged dextrans of distinct molecular weights in three sets: Texas Red®-tagged of 3,000 Da (m=4), Texas Red®-tagged of 70,000 Da (n=4), or tetramethylrhodamine-tagged of 2,000,000 Da (l=4). In addition, two controls were studied with the first mouse sonicated post-microbubble injection but not injected with dextran, and the second mouse FUS-targeted with microbubbles and 3,000 Da dextran injected but no sonication.

Approximately 20 min after dextran injection, in order to allow for its circulation throughout the vasculature, the mice were transcardially perfused with 30 ml of phosphate buffer saline (138 mM sodium chloride, 10 mM phosphate, pH 7.4) and 60 ml of 4% paraformaldehyde. The mouse head was severed, the brain was then extracted from the skull and soaked in paraformaldehyde overnight. The brain was cyroprotected by soaking it in 10% sucrose for 30 minutes, 20% sucrose for 30 minutes, and 30% sucrose overnight. The brains were then embedded in O.C.T., frozen in a square mold, and then sectioned into 300 μm slices in either a horizontal or coronal orientation using a cryostat. A 300-μm thick section was chosen since it allowed for the analysis of the arteries and veins, which were determined to be contributing factors in drug deposition in our previous study.

Images were acquired using an inverted light and fluorescence microscope (IX-81; Olympus, Melville, N.Y.) with rhodamine optics. All images were analyzed identically in MetaMorph (Molecular Devices, Downingtown, Pa.) and post-processed in MATLAB.

The fluorescence intensity range was varied so that the right hippocampus was barely visible and the left hippocampus was not completely saturated with fluorescence. This range was applied to all images so that they could be compared with one another. In order to obtain quantitative data, the left and right hippocampi were traced, the mean spatial fluorescence intensity in that region was measured, and then the ratio of fluorescence of the left over the right hippocampus was calculated. This procedure was repeated for all mice.

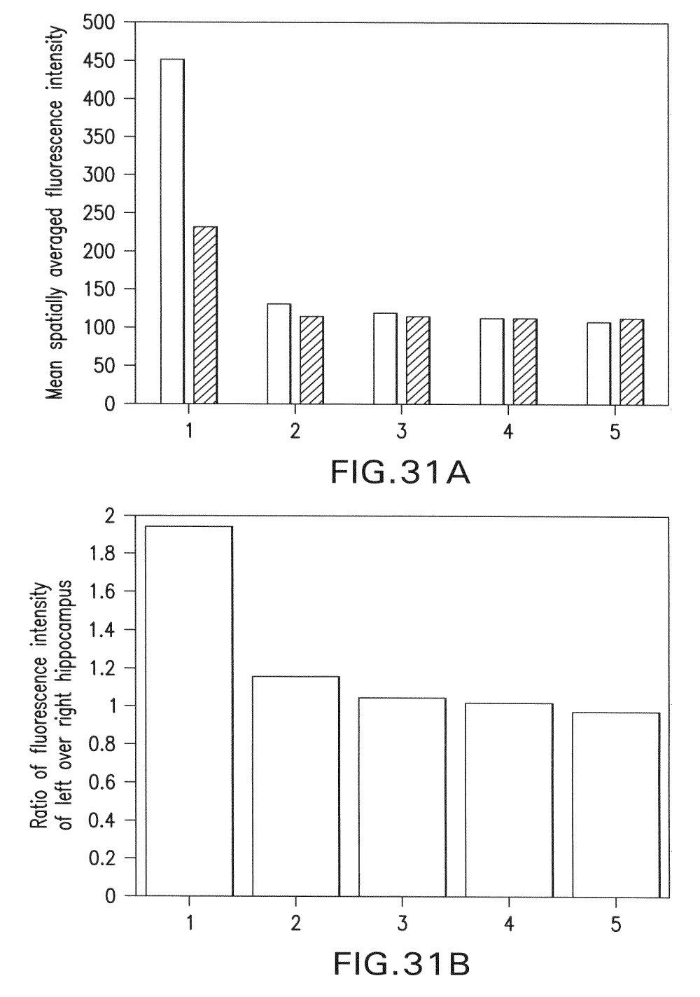

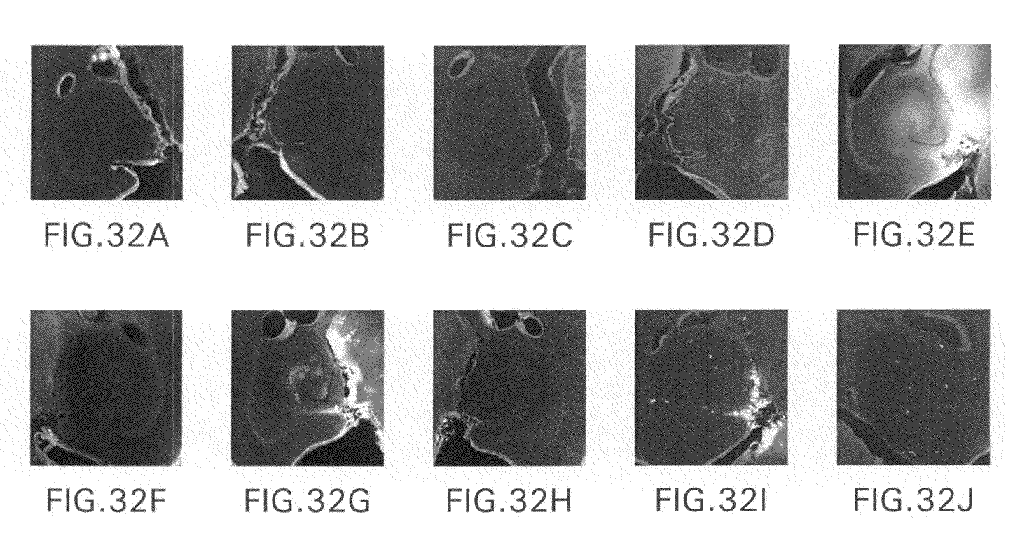

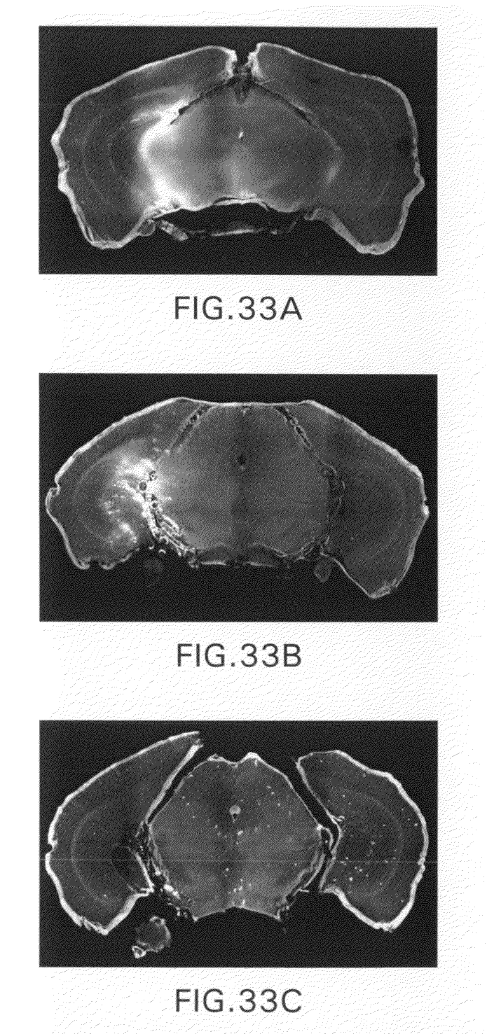

The spatial distribution of fluorescent markers delivered through the FUS-induced BBB openings was investigated with serial sections of the brains at defined planes. The BBB was expected to open only within the targeted region. The FUS transducer's focal beam had a 13.0 mm axial length and a 1.32 mm diameter, and was positioned to overlap the hippocampus, PCA, and part of the thalamus region bordering the hippocampus. In the absence of sonication or in the absence of i.v. injected fluorescent markers, no differences in fluorescence intensity were registered between the contralateral hemispheres, indicating no BBB opening (FIGS. 31 (A and B), and 32(A,B,C,D)). FIGS. 31(A-B) are based on the molecular weight of the molecule. 1:3,000 Da dextrans, 2:70,000 Da dextrans, 3:2,000,000 Da dextrans, 4: control 1, 5: control 2. FIGS. 32(A-J) illustrate horizontal sections of the left (targeted; FIGS. 32(A,C,E,G,I)) and right (control; FIGS. 32(B, D, F, H, J)) hippocampus of five mouse brains. The first mouse (FIGS. 32(A,B)) was not sonicated, but injected with 30,000 Da dextrans. The second mouse (FIG. 32(C,D)) was sonicated, but no dextran was injected. In these two mice, no fluorescence was observed. Fluorescence was observed in the targeted hippocampus when 3,000 (FIGS. 32(E,F)), 70,000 (FIG. 32(G,H), and 2,000,000 (FIG. 32(I,J) Da dextrans were injected after sonication. The application of FUS and microbubbles resulted in a marked transfer of fluorescent dextran markers at 3,000 and 70,000 Da to the left hippocampus (FIG. 31). Intravenous injection of 3,000 Da dextrans exhibited a significant increase in fluorescence intensity throughout the left hippocampus and part of its immediate surrounding regions (i.e., the thalamus). Less fluorescence intensity was observed at 70,000 Da, but was still significantly greater in the left hippocampus than in the right hippocampus. In addition, instead of being uniformly distributed, the fluorescence was concentrated to certain regions of the mouse brain within the targeted focus, i.e., larger vessels (PCA, longitudinal and transverse hippocampal arteries), the dentate gyrus, and the thalamus region bordering the hippocampus. In three out of four mice, no significant fluorescence by 2,000,000 Da dextrans was observed in the left hippocampus (FIG. 31), and, instead, was observed at the left PCA and its initial segments of branches (i.e., transverse and longitudinal hippocampal arteries) (FIG. 32. IJ). In one of the mice injected with 2,000,000 Da dextrans, localized regions of fluorescence were observed in other regions within the region targeted, i.e., transverse hippocampal arteries, dentate gyrus, etc. (FIGS. 33(A-C)). FIGS. 33(A-C) illustrate coronal sections of the hippocampi of three mouse brains. Fluorescence was observed in the targeted hippocampus when 3,000 (FIG. 33(A)) and 70,000 (FIG. 33(B)) dextrans were injected after sonication. No significant fluorescence was observed in the hippocampi after 2,000,000 Da dextrans were injected after sonication (FIG. 33(C)).

According to this example, BBB opening was shown to noninvasively, transiently, and locally deliver molecules at various molecular weights (3,000, 70,000, and 2,000,000 Da) in the brain of mice in vivo. The size of BBB opening may vary within different regions. As a result of this nonuniform opening and the different diffusion rates, smaller molecules were more uniformly deposited throughout the hippocampus than larger molecules. Finally, the BBB opening was shown to close within a day in a spatially significant manner.

The procedures for opening the BBB in a subject as described herein may be used in connection with cultured cells or on subjects, such as humans. For use in human subjects, the noninvasive FUS technique on the intact skull is a requirement. Targeting techniques may include locating anatomical landmarks as discussed above or using known stereotactic procedures. The increased thickness of the human skull when compared with the mouse skull may require the use of a lower frequency transducer, the frequency of 1.525 MHZ may be lowered to about 10-200 kHz. The bolus of microbubbles or contrast agents to be used would be adjusted to account for the larger mass in the case of human subjects.

It will be understood that the foregoing is only illustrative of the principles of the invention, and that various modifications can be made by those skilled in the art without departing from the scope and spirit of the invention. Moreover, features of embodiments described herein may be combined and/or rearranged to create new embodiments.

What is claimed is:

1. A method for opening the blood-brain barrier in the brain of a subject comprising:
targeting a region of the brain of a subject for opening; and
applying an ultrasound beam by a single-element transducer through the skull of the subject to the targeted region to open the blood-brain barrier in the brain of the subject.

2. A method according to claim 1, wherein applying an ultrasound beam by a single-element transducer through the skull of the subject comprises applying an ultrasound beam by a plurality of single-element transducers through the skull of the subject.

3. A method according to claim 1, wherein applying an ultrasound beam comprises applying a focused ultrasound beam.

4. A method according to claim 1, further comprising administering a molecule to the subject for passage across the blood-brain barrier.

5. A method according to claim 1, wherein targeting a region of the brain comprises locating at least one anatomical landmark of the subject.

6. A method for opening the blood-brain barrier in the brain of a subject comprising:
targeting vasculature adjacent selected brain tissue of a subject for opening; and
applying an ultrasound beam through the skull of the subject to the targeted vasculature to open the blood-brain barrier in the brain of the subject.

7. A method according to claim 6, wherein applying an ultrasound beam through the skull of the subject comprises generating an ultrasound beam having a frequency of about 1.525 MHz.

8. A method according to claim 6, wherein the ultrasound beam comprises a focus and wherein applying an ultrasound beam through the skull of the subject comprises generating an ultrasound beam having an acoustic pressure at the focus of about 0.5 to 2.7 MPa.

9. A method according to claim 6, wherein applying an ultrasound beam through the skull of the subject comprises generating an ultrasound beam having a burst rate of about 10 Hz.

10. A method according to claim 6, wherein applying an ultrasound beam through the skull of the subject comprises generating an ultrasound beam having a burst duration of about 20 ms.

11. A system for opening the blood-brain barrier in the brain of a subject comprising:
a targeting assembly for targeting a region of the brain of a subject for opening the blood-brain barrier thereof; and
a transducer for applying an ultrasound beam through the skull of the subject to the targeted region to open the blood-brain barrier in the brain of the subject.

12. A system according to claim 11, wherein the transducer comprises a single-element focused transducer.

13. A system according to claim 11, further comprising an introducer for introducing a molecule into the bloodstream of the subject for passage through the blood-brain barrier.

14. A system according to claim 11, wherein the targeting assembly comprises an ultrasound transducer.

15. A system according to claim 11, wherein the targeting assembly further comprises one or more members for placement on an anatomical landmark of the subject.

16. A system for opening the blood-brain barrier in the brain of a subject comprising:
means for targeting a region of the brain of a subject for opening; and
means for applying an ultrasound beam through the skull of the subject to the targeted region to open the blood-brain barrier in the brain of the subject.

17. A system according to claim 16, wherein the means for applying an ultrasound beam comprises a single-element focused transducer.

18. A system according to claim 16, wherein the means for applying an ultrasound beam is adapted to generate a focused ultrasound beam having a frequency of about 1.525 MHz.

19. A system according to claim 16, wherein the focused ultrasound beam comprises a focus and wherein the means for applying a focused ultrasound beam is adapted to generate a focused ultrasound beam having an acoustic pressure at the focus of about 0.5 to 2.7 MPa.

20. A system according to claim 16, wherein the focused ultrasound beam comprises a focus and wherein the means for applying a focused ultrasound beam is adapted to generate a focused ultrasound beam having a burst rate of about 20 Hz.

21. A system for delivering a molecule through the blood-brain barrier in the brain of a subject comprising:
a targeting assembly for targeting a region of the brain of a subject;
a transducer for applying an ultrasound beam through the skull of the subject to the targeted region to open the blood-brain barrier in the brain of the subject; and
an introducer for delivering a molecule through the blood-brain barrier of the subject.

22. A system according to claim 21, wherein the transducer comprises a single-element focused transducer.

23. A system according to claim 21, wherein the introducer comprises a needle.

24. A system according to claim 21, wherein the introducer comprises a catheter.

25. A system according to claim 21, wherein the molecule comprises a drug.
